(19) United States
(12) Patent Application Publication
(10) Pub. No.: US 2011/0217281 A1
(43) Pub. Date: Sep. 8, 2011

(54) METHODS FOR DIAGNOSIS AND PROGNOSIS OF CANCER

(76) Inventors: Jeffrey M. Trent, Phoenix, AZ (US); Michael Bittner, Phoenix, AZ (US); Edward R. Daugherty, College station, TX (US); Yuan Jaing, Bethesda, MD (US); Ronaldo Fumio, Sao Paulo (BR)

(21) Appl. No.: 13/055,132

(22) PCT Filed: Jul. 21, 2009
(86) PCT No.: PCT/US09/04254

§ 371 Date: May 11, 2011

Related U.S. Application Data
(60) Provisional application No. 61/082,426, filed on Jul. 21, 2008.

(51) Int. Cl. A61K 38/46; C12Q 1/42; G01N 33/573; C40B 30/00; A61K 31/7088; A61K 38/22; A61P 35/00

(52) U.S. Cl. 424/94.6; 435/21; 435/7.4; 506/7; 514/44 R; 514/9.7

(57) ABSTRACT

A method for diagnosing an early stage melanoma which includes detecting the expression level of dual specificity phosphatase 1 protein (DUSP1) and a method for determining a prognosis for a subject having melanoma which includes detecting the expression level of DUSP1 are disclosed. Also disclosed are pharmaceutical compositions and kits comprised by these methods.

### FIELD

Methods for diagnosis of cancers, such as melanoma, are provided. Prognosis methods for cancers, such as melanoma, are provided. In one embodiment, the methods involve detecting the expression level of DUSP1 in a subject sample.

### BACKGROUND

There is a steady increase in the incidence of malignant melanomas in the United States. Since the prognosis for melanoma worsens with lesion thickness, early diagnosis and complete resection of the melanoma are essential for containing the malignancy. The key to improved survival for subjects with melanoma is early detection. However, although early diagnosis is vital, it is also problematic. Studies have reported unsatisfactory diagnostic accuracy using histopathology and high inter-physician variability.

A dysplastic nevus is an atypical mole that appears different from common moles on the skin. Typically, dysplastic nevi are larger than ordinary moles and have abnormal and indistinct borders with non-uniform color ranging from pink to brown. They is found anywhere on the skin and although they are usually flat, parts of them may raise above the skin surface. Dysplastic nevi are more likely to develop into melanoma than ordinary moles.

The clinical and histological definitions for dysplastic nevi are still evolving. Numerous definitions and criteria have been proposed including the term “dysplastic nevi” for histologically abnormal nevi. Unfortunately, when clinically abnormal nevi are evaluated histologically, some studies have shown a lack of concordance with some clinically abnormal nevi showing no dysplastic features and some normal-appearing nevi showing dysplastic features. Dysplastic nevi overlap clinically with the widely used ABCDE criteria (Asymmetry, Border irregularity, Color variation, Diameter>6 mm, and Enlargement or Evolution) for melanoma. Similarly, Spitz nevi (a benign juvenile melanoma with atypical melanocytes) are known melanoma simulators at both the architectural and cytologic level. Therefore, their differentiation from melanomas may also pose significant difficulties.

A number of parameters have been cited as having prognostic significance for melanoma including vertical growth phase, thickness, depth of invasion, ulceration, angiolymphatic invasion, satellitosis, mitotic activity, host response, and regression. However, evaluation of these parameters can easily vary by physician.

The DUSP1 (CL100, MKP-1) is the prototypical member of a family of dual specificity (threonine/tyrosine) phosphatases that dephosphorylate MAP-kinases (Alessi et al., Oncogene 8(7):2015-2020, 1993). The DUSP1 phosphatase is known to act on p38 MAP kinase, a pivotal agent in malignant melanoma, that is significant in UVA and UVB induction of NMSC (Smalley, K. S., Int J Cancer 104(5):527-532, 2003). DUSP1 is activated and stabilized by the phosphorylation activity of MAPK and plays a regulating role in dampening the MAPK activity. DUSP1 serves as an effective antagonist to a variety of processes stimulated by activated MAPK, including both proliferation and apoptosis (Smalley, supra). This antagonism makes it an important gene to consider in oncogenic processes, as mutations in the Ras/Raf signaling pathway, that lead to constitutive MAPK activation, are common in a wide variety of cancers, including melanoma (Davies et al., Nature 417(6892):949-954, 2002; Bos, J. L. Cancer Res 49(17):4682-4589, 1989).

DUSP1 is also a feedback modulator of excessive MAPK activation, as its production is stimulated by long term MAPK activation, and its phosphorylation by MAPK renders it more resistant to proteolytic degradation (Brondello et al., Science 286(5449):3514-2517, 1999). DUSP1 acts as a tumor suppressor and is the last safeguard against MAPK tumorogenic activity. When DUSP1 is turned off or is absent, a cancer becomes much more aggressive/invasive. DUSP is an antagonist to both proliferation and apoptosis. As a result, its effect on tumor survival is unpredictable and will depend on context.

There is a need for a method for diagnosing an early stage melanoma as well as a better defined and less variable method for determining the prognosis for a subject with melanoma. Based on its characteristics, DUSP1 seems to be a useful object for this purpose.

### SUMMARY

In one embodiment, provided is a method for diagnosing a skin cancer in a subject comprising detecting the expression level of dual specificity phosphatase 1 protein (DUSP1).

The method involves detecting the expression level of DUSP1 in a sample from the subject and comparing the DUSP1 expression level to a predetermined index of DUSP1 expression. In one embodiment, the comparison provides a determination for the presence or absence of a skin cancer. In one embodiment, the expression of DUSP1 is detected for the diagnosis of an early stage melanoma. In one embodiment, the expression level of DUSP1 is detected by immunohistochemical staining techniques.

In another embodiment, provided is a prognostic method for cancer survivability in a subject diagnosed with skin cancer. The method involves detection of the DUSP1 expression level in a tumor tissue or cancer cell and comparison of the expression level of the DUSP1 with an index of DUSP1 expression that corresponds to a survivability assessment. In some embodiments, the survivability assessment is for long-term survivability. In some embodiments, the survivability assessment is for short-term survivability. It has been found herein that a subject diagnosed with skin cancer having increased levels of DUSP1 exhibits a longer term of survival than a skin cancer subject that does not have an increased level of DUSP1. Conversely, it has been found herein that an individual having significantly reduced levels of DUSP1 exhibits a significantly shorter term of survival and thus requires much more aggressive therapeutic treatment. In one embodiment, the expression of DUSP1 is detected for the prognosis of an early stage melanoma. In one embodiment, the expression level of DUSP1 is detected by immunohistochemical staining techniques.

In yet another embodiment, provided is a method of treating skin cancer comprising administering to a patient in need of such treatment an effective amount of DUSP1 or an active fragment thereof, a nucleic acid that encodes a protein comprising DUSP1 or an active fragment thereof, an analog or a prodrug thereof. In one embodiment, provided is a method of treating cancer comprising administering to a skin cancer patient an effective amount of DUSP1 and one or more additional therapeutic agents. In one embodiment, the additional therapeutic agent is capable to enhance a degree of expression of DUSP1. In one embodiment, the skin cancer is an early stage melanoma.

In another embodiment, provided is a pharmaceutical composition for treating skin cancer. In one embodiment, the pharmaceutical composition comprises a DUSP1 or an active fragment thereof, a nucleic acid that encodes a protein comprising DUSP1 or an active fragment thereof, an analog or a prodrug thereof, and a pharmaceutically acceptable carrier. In another embodiment, the pharmaceutical composition comprises a DUSP1 and one or more additional therapeutic agents. In one embodiment, the additional therapeutic agent is capable of enhancing the degree of expression of DUSP1.

In another embodiment, provided are kits comprising a reagent for detection of DUSP1 expression levels, a compilation comprising DUSP1 expression level indices that have been predetermined to correlate with the presence or absence of skin cancer, assay components and components for collection of a sample. In another embodiment, a kit comprising a reagent for detection of DUSP1 expression levels, a compilation comprising DUSP1 expression level indices that have been predetermined to correlate with cancer survivability, assay components and components for collection of a sample.

### BRIEF DESCRIPTION OF THE DRAWINGS

FIG. 1 shows a logical truth table for DUSP1 predictions based on the expression levels of one set of three genes relative to the expression level of DUSP1. It demonstrates the best predictive rules that could be inferred for DUSP1's behavior using the behavior of 3 other genes as predictors. For the estimation of optimal rules the discretized expression behavior (abundance significantly greater than the reference=+1, significantly less=−1 and not significantly changed=0) of a set of 580 genes profiled in 31 melanoma samples were used to construct all possible 3 gene predictors for all possible genes. The best logical predictive rules were constructed for all predictive sets. The resulting tables were searched for situations where there was determinate prediction within a set of samples defined by a single gene's state.

FIGS. 2a and 2b show dependency of melanoma patients' survival rate from expression levels of DUSP1.

FIG. 3 shows the relationship of DUSP1 to MAPK in the RAS/RAF pathway.

FIG. 4 shows intense immunohistochemical staining for DUSP1 at the invading edges of radial growth phase melanoma.

FIG. 5 shows heavy immunohistochemical staining for DUSP1 in two small tumors from a long-term survivor of metastatic melanoma which were removed at year 15 and again at year 17 during the long-term survivor's 17 years of living with Stage 1V melanoma.

FIG. 6a shows the IHC results for a melanoma sample positively staining for DUSP1 using Universal Alkaline Phosphatase RED (Ventana Medical Systems, Tucson, Ariz.) as described in Example 1. FIG. 6b shows the IHC results for a melanoma sample negative for DUSP1 staining using Universal Alkaline Phosphatase RED (Ventana Medical Systems, Tucson, Ariz.) as described in Example 1.

FIG. 7 is a KM Graph Showing DUSP1 Expression vs. Percent Survival over 10 years.

FIG. 8 is a microarray analysis showing determinant behavior of genes in the presence and absence of DUSP1 expression.

### DETAILED DESCRIPTION

General Definitions

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. It should also be noted that use of “or” means “and/or” unless stated otherwise. Furthermore, use of the term “including” as well as other forms, such as “include,” “includes,” and “included” is not limiting.

The term “subject,” “patient” or “individual” as used herein in reference to individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods provided herein, the mammal is a human.

The term “treating” and its grammatical equivalents as used herein include achieving a therapeutic benefit and/or a prophylactic benefit. By therapeutic benefit is meant eradication or amelioration of the underlying disorder being treated. Treating also refers to obtaining a desired pharmacologic and/or physiologic effect. The effect may be prophylactic in terms of completely or partially preventing a condition or disease or symptom thereof and/or may be therapeutic in terms of a partial or complete cure for a condition or disease and/or adverse affect attributable to the condition or disease. “Treatment,” thus, for example, covers any treatment of a condition or disease in a mammal, particularly in a human, and includes: (a) preventing the condition or disease from occurring in a subject which may be predisposed to the condition or disease but has not yet been diagnosed as having it; (b) inhibiting the condition or disease, such as, arresting its development; and (c) relieving, alleviating or ameliorating the condition or disease, such as, for example, causing regression of the condition or disease. By way of example only, in a cancer patient, therapeutic benefit may include eradication or amelioration of the underlying cancer. Also, a therapeutic benefit may be achieved with the eradication or amelioration of one or more of the physiological symptoms associated with the underlying disorder such that an improvement is observed in the patient, notwithstanding the fact that the patient may still be afflicted with the underlying disorder. For prophylactic benefit, a method may be performed on, or a composition administered to a patient at risk of developing cancer, or to a patient reporting one or more of the physiological symptoms of such conditions, even though a diagnosis of the condition may not have been made. In some instances, treating means stasis (i.e., that the disease does not get worse) and survival of the patient is prolonged. A dose to be administered depends on the subject to be treated, such as the general health of the subject, the age of the subject, the state of the disease or condition, the weight of the subject, the size of a tumor, for example.

It is understood that the genes and/or proteins described herein are inclusive of allelic variant isoforms, synthetic nucleic acids and/or proteins, nucleic acid and/or proteins isolated from tissue and cells, and modified forms thereof. It is also understood that the genes and/or proteins described herein are also known to exist in various forms, including variants and mutants, and are contemplated herein. The genes and/or proteins described herein further include nucleic acid sequences and/or amino acid sequences having at least 65% identity with the gene or protein to be detected and are included within embodiments described herein. In various aspects, DUSP1 expression levels are determined either by determining a level of nucleic acid, protein or protein activity in a sample.

The term “biological sample” is intended to include tissues (including, but not limited to, tissue biopsies), cells, biological fluids and isolates thereof, isolated from a subject, as well as tissues, cells and fluids present within a subject.

The term “modulate” or “modulating” or any variation of the term in the context of any methods disclosed herein means increasing DUSP1 expression (e.g., mRNA or protein level) and/or increasing DUSP1 protein activity. In other embodiments, “modulate” means decreasing DUSP1 expression and/or decreasing DUSP1 protein activity.

As used herein, the term “expression vector or construct” means any type of genetic construct containing a nucleic acid coding for a gene product in which part or all of the nucleic acid encoding sequence is capable of being transcribed. The transcript is translated into a protein, but it need not be. Thus, in certain embodiments, expression includes both transcription of a gene and translation of a RNA into a gene product. In other embodiments, expression only includes transcription of the nucleic acid, for example, to generate antisense constructs.

As used herein, the terms “engineered” and “recombinant” cells or host cells are intended to refer to a cell into which an exogenous DNA segment or gene, such as a cDNA or gene encoding a protein has been introduced. Therefore, engineered cells are distinguishable from naturally occurring cells which do not contain a recombinantly introduced exogenous DNA segment or gene. Engineered cells are cells having a gene or genes introduced through the hand of man. Recombinant cells include those having an introduced cDNA or genomic gene, and also include genes positioned adjacent to a promoter not naturally associated with the particular introduced gene.

The term “purified” as used herein, is intended to refer to a proteinaceous composition, isolatable from mammalian cells or recombinant host cells, wherein at least one polypeptide is purified to any degree relative to its naturally-obtainable state, i.e., relative to its purity within a cellular extract. A purified polypeptide therefore also refers to a wild-type or modified polypeptide free from the environment in which it naturally occurs.

Where the term “substantially purified” is used, this will refer to a composition in which the specific polypeptide forms the major component of the composition, such as constituting about 50% of the proteins in the composition or more.

A polypeptide that is “purified to homogeneity,” as provided herein, means that the polypeptide has a level of purity where the polypeptide is substantially free from other proteins and biological components. For example, a purified polypeptide will often be sufficiently free of other protein components so that degradative sequencing is performed.

The term “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier must be “acceptable” in the sense of being compatible with the other ingredients of the formulation and not injurious to a subject to whom it is administered. Nor should an acceptable carrier alter the specific activity of the subject compounds.

The term “pharmaceutically acceptable” refers to molecular entities and compositions that are physiologically tolerable and do not typically produce an allergic or similar untoward reaction, such as gastric upset, dizziness and the like, when administered to a human.

The term “unit dose” when used in reference to a therapeutic composition refers to physically discrete units suitable as unitary dosage for humans, each unit containing a predetermined quantity of active material calculated to produce the desired therapeutic effect in association with the required diluent; i.e., carrier, or vehicle.

A “therapeutically effective amount” as used herein, is an amount that achieves at least partially a desired therapeutic or prophylactic effect in an organ or tissue.

The term “packaging material” refers to a physical structure housing the components of the kit. The packaging material can maintain the components sterilely, and is made of material commonly used for such purposes (e.g., paper, corrugated fiber, glass, plastic, foil, ampules, etc.).

DUSP1

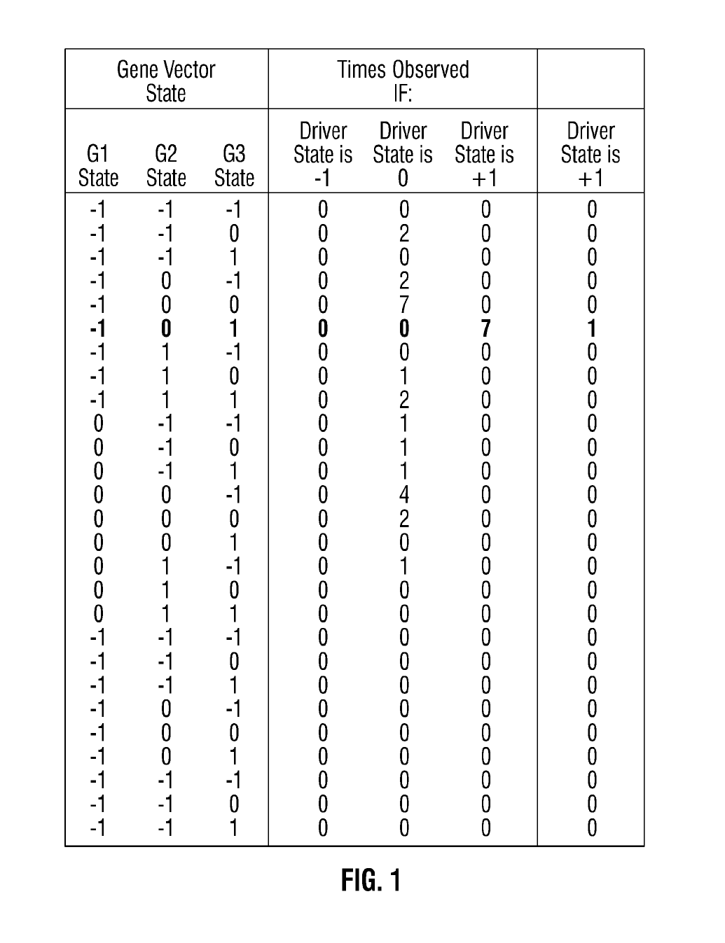

Genes showing broad regulatory activity and acute, intermittent control often play key roles in cancer. Algorithms have been developed that make possible the identification of such genes within expression profiles. An analysis that characterizes the predictive behavior of a set of 1, 2 or 3 genes for a single gene target has been used as a primary tool. The analysis provided a calculated, normalized value for the extent of co-determination that is achieved using the best logical rule that is formulated to predict the discretized expression level of the single gene, given the discretized expression levels of the set of genes (Kim et al., Genomics 67(2):201-209, 2000). This value is a sensitive reporter of small increments of tight control even in a set of samples where the tight control is exerted only in a small fraction of the total number of samples profiled. An exhaustive search (1.8×10¹⁰ possible predictive relationships tested) for such genes was carried out on 580 genes from 31 melanoma samples (Bittner et al., Nature 406(6795):536-540, 2000). Using the samples from the search, the inventors have identified twenty-seven genes to show absolutely determinate behavior when the gene, DUSP1, was highly expressed, but random behavior in the absence of DUSP1 expression. A logical truth table illustrating this phenomenon for the expression levels of one set of three genes relative to the expression level of DUSP1 is shown in FIG. 1.

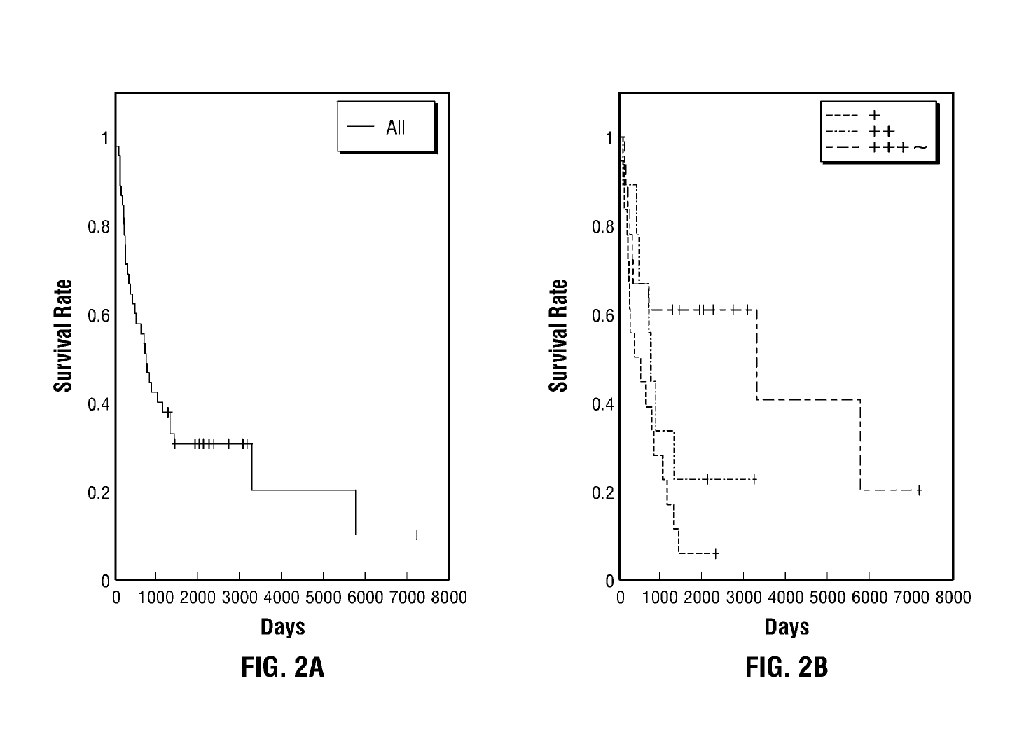

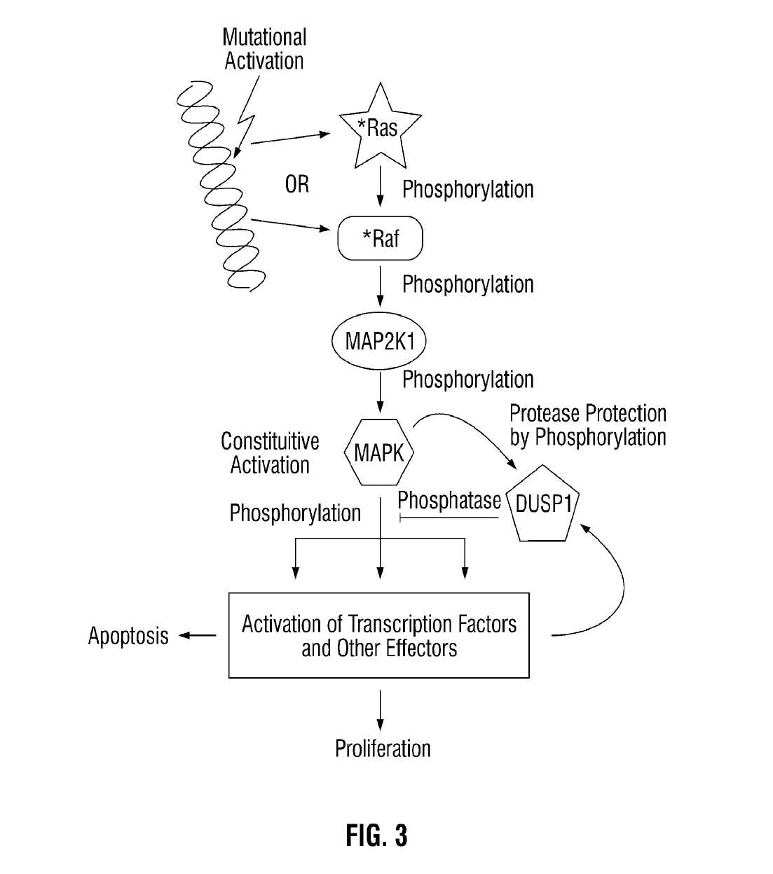

Analysis of DUSP1 expression in subjects having cancer with known clinical outcomes identified a correlation between levels of DUSP1 expression and survivability, where increasing expression of DUSP1 corresponds with increased survival time. A retrospective comparison of survival information using a small tissue microarray (TMA) containing 54 melanoma tumors with known outcome was conducted. It was shown that the Kaplan Meier plots for all tumors and for tumors segregated on the basis of intensity of DUSP1 immunostaining clearly demonstrated that high levels of DUSP1 protein are correlated with increased survival (p<0.001), as shown in FIG. 2. Increased DUSP1 expression can provide a prognosis of long-term survival, whereas decreased DUSP1 expression can provide a prognosis of short-term survival. Thus, the detection of DUSP1 expression levels is utilized to provide a prognosis for survival in a patient with cancer. Furthermore, the ability to provide a prognosis regarding survivability can also assist in the determination of therapy for a subject. For example, when a prognosis is for short-term survivability, a subject may elect more aggressive and/or experimental therapies. Conversely, a subject with prognosis of long-term survivability may elect standard-of-care therapies or therapies with lesser side-effects, etc.

As DUSP1 expression is induced by constitutive MAP kinase signaling, this gene is induced early in many skin cancers including melanomas. As such, DUSP1 is an early indication of melanoma and acts as a differentiation marker for dysplastic nevi. Simply, DUSP1 presence in dysplastic nevi is an indication that the RAS/RAF/MAPK pathway is active and the cells are already in a transformed state. Furthermore, as DUSP1 is an attenuator of MAPK activity, absence of DUSP1 in confirmed melanoma is a prognostic indicator for melanoma aggressiveness. Accordingly, if DUSP1 is not expressed in confirmed melanoma, a patient has a poor prognosis and a short life expectancy, whereas if DUSP1 is expressed in confirmed melanoma, then the prognosis is good with a much longer life expectancy.

The early induction of DUSP1 expression is monitored or measured for a more definitive test of malignancy than the current use of depth of invasion by the methods described herein to provide a diagnosis for skin cancer. There are several types of cancer that start in the skin. The most common types are basal cell carcinoma and squamous cell carcinoma, which are non-melanoma skin cancers. Actinic keratosis is a skin condition that sometimes develops into squamous cell carcinoma. Non-melanoma skin cancers rarely spread to other parts of the body. Melanoma, the rarest form of skin cancer, is more likely to invade nearby tissues and spread to other parts of the body.

Skin cancers to be diagnosed include, but are not limited to, basal cell carcinomas, squamous cell carcinomas, melanomas such as malignant melanomas, cutaneous melanomas or intraocular melanomas, Dermatofibrosarcoma protuberans, Merkel cell carcinoma, neuroectodermal tumor, lymphatic epithelial tumors and Kaposi's sarcoma. In one embodiment, the skin cancer is melanoma.

As described herein, DUSP1 is a gene identified as differentially expressed in cancer. Cancers contemplated within the present application include, but are not limited to, lung cancer, a skin cancer, a breast cancer, a colon cancer, a colorectal cancer, a head and neck cancer, a liver cancer, a prostate cancer, an ovarian cancer, or a uterine cancer, or metastases of any thereto.

Because DUSP1 is an antagonist to both proliferation and apoptosis, DUSP1 expression can extend survival by greatly reducing the accumulation of tumor burden and limiting the rate of tumor evolution. Furthermore, its continued presence correlates with protection against apoptosis induced by either UV or chemotherapy (Denkert et al., Int J Cancer 102(5):507-513, 2002). In skin cancer, for example in melanoma, where the high constitutive levels of other anti-apoptotic molecules drive the escape from apoptosis, the anti-proliferative action of DUSP1 leads to an uncomplicated improvement in survival with clear benefits toward prevention efforts in melanoma.

Additionally, the DUSP1 is a beneficial therapeutic for melanoma either alone or in combination with other therapeutic compositions. Methods of screening a library of compounds including libraries derived from combinatorial chemistry, antibody fragments, peptides, siRNA, or any other of potential therapeutic compounds are well known in the art and is adapted to screen for those compounds that modulate DUSP1 expression.

Diagnosis and Prognosis of Cancer

The present disclosure relates generally to the diagnosis of cancer and/or the prognosis for cancer survivability in a subject by detecting the expression of DUSP1 in a sample from the subject and comparing the expression level to a predetermined index of DUSP1 expression. In one embodiment, a method of screening is provided comprising determining DUSP1 expression in a sample from a subject, comparing the expression levels to a predetermined index of DUSP1 expression, wherein the comparison provides a determination for the presence or absence of a cancer.

In another embodiment, a prognostic method is provided wherein a DUSP1 expression in a tumor tissue or cancer cell is compared with an index of DUSP1 expression that corresponds to a survivability assessment. In some embodiments, the survivability assessment is for long-term survivability. In some embodiments, the survivability assessment is for short-term survivability.

Cancers contemplated within the present application include, but are not limited to, lung cancer, a skin cancer, a breast cancer, a colon cancer, a colorectal cancer, a head and neck cancer, a liver cancer, a prostate cancer, an ovarian cancer, or a uterine cancer, or metastases of any thereto. In one embodiment, a cancer as contemplated in methods and compositions provided herein is melanoma.

As described herein, DUSP1 has been found to correlate with cancer detection as well as determination of the prognosis for a subject having cancer. The prognosis for survival increases with increasing DUSP1 expression levels in the cancer samples from the subjects, thus expression level indicies of DUSP1 expression levels is constructed based on identified correlations between DUSP1 expression levels and known clinical outcomes. Such indicies provide a comparison standard for providing a prognosis based on the DUSP1 expression levels obtained from a subject to be tested.

In one embodiment, provided herein is a method for screening for skin cancer comprising detecting the expression of DUSP1 in a sample from a subject and comparing the expression of DUSP1 to level of DUSP1 expression predetermined to correlate with the absence or presence of skin cancer. Skin cancers include, but are not limited to, basal cell carcinomas, squamous cell carcinomas, melanomas such as malignant melanomas, cutaneous melanomas or intraocular melanomas, Dermatofibrosarcoma protuberans, Merkel cell carcinoma and Kaposi's sarcoma. In one embodiment, the skin cancer is melanoma. In another embodiment, the expression of DUSP1 is detected by molecular techniques to detect mRNA levels. In yet another embodiment, the expression of DUSP1 is detected by protein detection techniques. Protein detection techniques include, but are not limited to, western blots, protein arrays, immunohistochemistry, ELISA, fluorescent-activated cell sorting, flow cytometry, and mass spectrometry.

In another embodiment, provided herein is a method for providing a prognosis of survivability for a subject having skin cancer. The expression of DUSP1 is detected in a skin cancer sample from a subject and compared to an expression level index of DUSP1 that is predetermined to correlate with survivability. The comparison then allows for a prognosis of survivability to be obtained based on the predetermined expression level index.

The expression level index is created using various measurement standards. Examples of biomarker measurement techniques are described below. The predetermined expression level index is created by assaying skin cancer samples from numerous subjects with the same type of skin cancer for the expression of DUSP1. The DUSP1 expression levels are then correlated with the observed survivability/clinical outcome of the subjects. Increasing survivability (e.g., long-term survivability vs. short-term survivability) corresponds to increasing percentages of DUSP1 expressing cells on the predetermined DUSP1 expression level index.

In one embodiment, a DUSP1 expression level index is determined by assaying the percentage of cells in a skin cancer sample that are positive for DUSP1 expression. The DUSP1 expression level index values that correlate with increased survivability range from about 5% to 100%, 10% to 100%, 15% to 100%, 20% to 100%, 25% to 100%, 30% to 100%, 35% to 100%, 40% to 100%, 45% to 100%, 50% to 100%, 55% to 100%, 60% to 100%, 65% to 100%, 70% to 100%, 75% to 100%, 80% to 100%, 85% to 100%, 90% to 100%, or 95% to 100% with all intervening integers included. In yet another embodiment, DUSP1 expression level index values that correlate with increased survivability range from 35% to 100%. In yet another embodiment, DUSP1 expression level index values that correlate with increased survivability range from 25% to 100%.

In another embodiment, a DUSP1 expression level index is determined by assaying the percentage of cells in a skin cancer sample that are positive for DUSP1 expression, and further categorizing or segregating the positive cells into semi-quantitative levels of expression based on the intensity of the staining. Exemplary categories or scale of positive DUSP1 levels of expression are in a range from 0 to 3, where 0 indicates no staining and 3 indicates an intense level of staining in a cell based on color and area stained within the cell.

In another embodiment, a DUSP1 expression level index is determined by assaying the percentage of cancer cells in a skin cancer sample that are negative for DUSP1 expression. The expression level index values for the percent cells negative for DUSP1 that correlate with reduced survivability range from about 5% to 100%, 10% to 100%, 15% to 100%, 20% to 100%, 25% to 100%, 30% to 100%, 35% to 100%, 40% to 100%, 45% to 100%, 50% to 100%, 55% to 100%, 60% to 100%, 65% to 100%, 70% to 100%, 75% to 100%, 80% to 100%, 85% to 100%, 90% to 100%, or 95% to 100% with all intervening integers included. In yet another embodiment, the expression level index values for the percent cells negative for DUSP1 that correlate with decreased survivability range from 35% to 100%. In yet another embodiment, the expression level index values for the percent cells negative for DUSP1 that correlate with decreased survivability range from 25% to 100%.

Expression levels of DUSP1 above the levels seen in cancerous cells with short-term survivability are known to confer protective effects against cancer including, but not limited to, treating the cancer, preventing the metastasis of the cancer, preventing the growth of the cancer, and increasing the death of the cancerous cells (e.g., increasing apoptosis of cancerous cells, etc.).

Provided herein are methods for the identification of compounds that can increase, up-regulate, and/or modulate DUSP1 expression. In one embodiment, provided herein are methods of identifying compounds for the treatment of skin cancers by assaying a skin cancer sample or cell line for DUSP1 expression before and after application of the test compound to the skin cancer sample or cell line to identify those compounds that increase the expression of DUSP1. In one embodiment, the skin cancer is melanoma. Additionally, when a compound increases the expression levels of DUSP1, such increased levels of DUSP1 expression is compared to a DUSP1 expression level index that has been predetermined to correlate with the presence or absence of melanoma. In another embodiment, the DUSP1 expression level is compared to a DUSP1 expression level index that has been predetermined to correlate with the survivability.

Assays and Methods of Detection

As described and used herein, biomarkers include both genes and proteins. The biomarker contemplated herein is DUSP1. In various aspects, DUSP1 expression levels are determined either by determining a level of nucleic acid, protein or protein activity in a sample.

Protein detection techniques and molecular techniques for detection of nucleic acids (e.g., DNA, RNA, mRNA, siRNA, etc.) are known in the art, and any combination or type are contemplated for use herein. Non-limiting examples of protein detection techniques and molecular techniques are described in more detail below and in the examples. Expression of DUSP1 may be assessed by any method used to detect nucleic acids and/or material derived from a nucleic acid template (e.g., proteins, fragments, etc.) used currently in the art. Examples of such methods include, but not limited to, microarray analysis, RNA in situ hybridization, RNAse protection assay, Northern blot, RTPCR, and QPCR. Other examples include, but not limited to, flow cytometry, immunohistochemistry, ELISA, Western blot, and immunoaffinity chromatography. Antibodies may be monoclonal, polyclonal, or any antibody fragment including a Fab, F(ab)₂, Fv, scFv, phage display antibody, peptibody, multispecific ligand, or any other reagent with specific binding to a target. Such methods also include direct methods used to assess protein expression including, but not limited to, HPLC, mass spectrometry, protein microarray analysis, PAGE analysis, isoelectric focusing, 2-D gel electrophoresis, and enzymatic assays. Samples from which expression may be detected include single cells, whole organs or any fraction of a whole organ, whether in vitro, ex vivo, in vivo, or post-mortem.

For assessment of tumor cell biomarker expression, patient samples containing tumor cells, or proteins or nucleic acids produced by these tumor cells, are used in methods described, for example, in U.S. Publication Number 20070065858, which is incorporated herein by reference in its entirety. The level of expression of the biomarker is assessed by assessing the amount (e.g., absolute amount or concentration) of the marker in a tumor cell sample, e.g., a tumor biopsy obtained from a patient, or other patient sample containing material derived from the tumor (e.g., blood, serum, urine, or other bodily fluids or excretions as described herein above). The cell sample is subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, tumor biopsies can also be subjected to post-collection preparative and storage techniques, e.g., fixation.

One can detect expression of biomarker proteins having at least one portion which is displayed on the surface of tumor cells which express it. One can determine whether a marker protein, or a portion thereof, is exposed on the cell surface. For example, immunological methods are used to detect such proteins on whole cells, or well known computer-based sequence analysis methods are used to predict the presence of at least one extracellular domain (i.e., including both secreted proteins and proteins having at least one cell-surface domain). Expression of a marker protein having at least one portion which is displayed on the surface of a cell which expresses it is detected without necessarily lysing the tumor cell (e.g., using a labeled antibody which binds specifically with a cell-surface domain of the protein).

Expression of biomarkers is assessed by any of a wide variety of well known methods for detecting expression of a transcribed nucleic acid or protein. Examples of such methods include, but not limited to, immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

Expression of a biomarker is assessed using an antibody (e.g., a radio-labeled, chromophore-labeled, fluorophore-labeled, or enzyme-labeled antibody), an antibody derivative (e.g., an antibody conjugated with a substrate or with the protein or ligand of a protein-ligand pair (e.g., biotin-streptavidin), or an antibody fragment (e.g., a single-chain antibody, an isolated antibody hypervariable domain, etc.) which binds specifically to a biomarker protein or fragment thereof, including a biomarker protein which has undergone either all or a portion of post-translational modifications to which it is normally subjected in the tumor cell (e.g., glycosylation, phosphorylation, methylation, etc.).

Expression of a biomarker can also be assessed by preparing mRNA/cDNA (i.e., a transcribed polynucleotide) from cells in a patient sample, and by hybridizing the mRNA/cDNA with a reference polynucleotide which is a complement of a biomarker nucleic acid, or a fragment thereof. cDNA can, optionally, be amplified using any of a variety of polymerase chain reaction methods prior to hybridization with the reference polynucleotide. Expression of one or more biomarkers can likewise be detected using quantitative PCR to assess the level of expression of one or more of the biomarkers. Alternatively, any of the known methods of detecting mutations or variants (e.g., single nucleotide polymorphisms, deletions, etc.) of a biomarker are used to detect occurrence of a biomarker in a patient.

In one embodiment, a mixture of transcribed polynucleotides obtained from the sample is contacted with a substrate having fixed thereto a polynucleotide complementary to or homologous with at least a portion (e.g., at least 7, 10, 15, 20, 25, 30, 40, 50, 100, 500, or more nucleotide residues) of a biomarker nucleic acid. If polynucleotides complementary to, or homologous with, are differentially detectable on the substrate (e.g., detectable using different chromophores or fluorophores, or fixed to different selected positions), then the levels of expression of a plurality of biomarkers are assessed simultaneously using a single substrate (e.g., a “gene chip” microarray of polynucleotides fixed at selected positions). When a method of assessing biomarker expression is used which involves hybridization of one nucleic acid with another, hybridization is performed under stringent hybridization conditions.

When a plurality of biomarkers is used in the methods described herein, the level of expression of each biomarker in a patient sample is compared with the normal level of expression of each of the plurality of biomarkers in non-cancerous samples of the same type, either in a single reaction mixture (i.e., using reagents, such as different fluorescent probes, for each biomarker) or in individual reaction mixtures corresponding to one or more of the biomarkers.

The level of expression of a biomarker in normal (i.e., non-cancerous) tissue is assessed in a variety of ways. This normal level of expression is assessed by assessing the level of expression of the biomarker in a portion of cells which appears to be non-cancerous, and then comparing the normal level of expression with the level of expression in a portion of the tumor cells. As further information becomes available as a result of routine performance of the methods described herein, population-average values for normal expression of the biomarkers is used. Alternatively, the normal level of expression of a biomarker is determined by assessing expression of the biomarker in a patient sample obtained from a non-cancer-afflicted patient, from a patient sample obtained from a patient before the suspected onset of cancer in the patient, from archived patient samples, and the like.

In one embodiment, a method for detecting the presence or absence of a biomarker protein or nucleic acid in a biological sample involves obtaining a biological sample (e.g., a tumor-associated body fluid) from a test subject and contacting the biological sample with a compound or an agent capable of detecting the polypeptide or nucleic acid (e.g., mRNA, genomic DNA, or cDNA). The detection methods can, thus, be used to detect mRNA, protein, cDNA, or genomic DNA, for example, in a biological sample in vitro as well as in vivo. In vitro techniques for detection of mRNA include, for example, Northern hybridizations and in situ hybridizations. Additional techniques for various nucleic acids include assays comprising various forms mass spectrometry (e.g., MALDI-TOF, etc.). Such mass determinations is utilized alone or in combination with additional molecular techniques such as sequencing reactions, chain termination reactions, primer extension reactions, and various known PCR reactions (e.g., RT-PCR, QC-PCR, etc.). In vitro techniques for detection of a biomarker protein include, but are not limited to, enzyme linked immunosorbent assays (ELISAs), mass spectrometry, Western blots, immunohistochemistry, immunoprecipitation and immunofluorescence. In vitro techniques for detection of genomic DNA include, for example, Southern hybridizations. In vivo techniques for detection of mRNA include, for example, polymerase chain reaction (PCR), Northern hybridizations and in situ hybridizations. Furthermore, in vivo techniques for detection of a biomarker protein include introducing into a subject a labeled antibody directed against the protein or fragment thereof. For example, the antibody is labeled with a radioactive marker whose presence and location in a subject is detected by standard imaging techniques.

A general principle of such diagnostic and prognostic assays involves preparing a sample or reaction mixture that may contain a biomarker, and a probe, under appropriate conditions and for a time sufficient to allow the biomarker and probe to interact and bind, thus forming a complex that is removed and/or detected in the reaction mixture. These assays are conducted in a variety of ways.

In another embodiment, a method to conduct such an assay involves anchoring the biomarker or probe onto a solid phase support, also referred to as a substrate, and detecting target biomarker/probe complexes anchored on the solid phase at the end of the reaction. In one embodiment of such a method, a sample from a subject which is to be assayed for presence and/or concentration of biomarker is anchored onto a carrier or solid phase support. In another embodiment, the reverse situation is possible, in which the probe is anchored to a solid phase and a sample from a subject is allowed to react as an unanchored component of the assay.

There are several established methods for anchoring assay components to a solid phase. These include, but not limited to, biomarker or probe molecules which are immobilized through conjugation of biotin and streptavidin. Such biotinylated assay components are prepared from biotin-NHS (N-hydroxy-succinimide) using techniques known in the art (e.g., biotinylation kit, Pierce Chemicals, Rockford, Ill.), and immobilized in the wells of streptavidin-coated 96-well plates (Pierce Chemical). In certain embodiments, the surfaces with immobilized assay components are prepared in advance and stored. Other suitable carriers or solid phase supports for such assays include any material capable of binding the class of molecule to which the biomarker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite. In order to conduct assays with the above mentioned approaches, the non-immobilized component is added to the solid phase upon which the second component is anchored. After the reaction is complete, uncomplexed components are removed (e.g., by washing) under conditions such that any complexes formed will remain immobilized upon the solid phase. The detection of biomarker/probe complexes anchored to the solid phase is accomplished in a number of methods outlined herein. In one embodiment, the probe, when it is the unanchored assay component, is labeled for the purpose of detection and readout of the assay, either directly or indirectly, with detectable labels discussed herein and which are well-known to one skilled in the art.

It is also possible to directly detect biomarker/probe complex formation without further manipulation or labeling of either component (biomarker or probe), for example by utilizing the technique of fluorescence energy transfer (i.e., FET, see for example, Lakowicz et al., U.S. Pat. No. 5,631,169; and Stavrianopoulos et al., U.S. Pat. No. 4,868,103). A fluorophore label on a donor molecule is selected such that, upon excitation with incident light of appropriate wavelength, its emitted fluorescent energy is absorbed by a fluorescent label on an acceptor molecule, which in turn is able to fluoresce due to the absorbed energy. Alternately, the donor protein molecule simply utilizes the natural fluorescent energy of tryptophan residues. Labels are chosen that emit different wavelengths of light, such that the acceptor molecule label is differentiated from that of the donor. Since the efficiency of energy transfer between the labels is related to the distance separating the molecules, spatial relationships between the molecules is assessed. In a situation in which binding occurs between the molecules, the fluorescent emission of the acceptor molecule label in the assay should be maximal. An FET binding event is conveniently measured through standard fluorometric detection means well known in the art (e.g., using a fluorimeter).

In another embodiment, determination of the ability of a probe to recognize a biomarker is accomplished without labeling either assay component (probe or biomarker) by utilizing a technology such as real-time Biomolecular Interaction Analysis (BIA; see, e.g., Sjolander, S, and Urbaniczky, C., 1991, Anal. Chem. 63:2338-2345 and Szabo et al., 1995, Curr. Opin. Struct. Biol. 5:699-705). As used herein, “BIA” or “surface plasmon resonance” refer to a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR), resulting in a detectable signal which is used as an indication of real-time reactions between biological molecules.

In another embodiment, analogous diagnostic and prognostic assays is conducted with biomarker and probe as solutes in a liquid phase. In such an assay, the complexed biomarker and probe are separated from uncomplexed components by any of a number of standard techniques, including but not limited to, differential centrifugation, chromatography, electrophoresis and immunoprecipitation. In differential centrifugation, biomarker/probe complexes is separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8): 284-7). Standard chromatographic techniques can also be utilized to separate complexed molecules from uncomplexed ones. For example, gel filtration chromatography separates molecules based on size, and through the utilization of an appropriate gel filtration resin in a column format, for example, the relatively larger complex is separated from the relatively smaller uncomplexed components. Similarly, the relatively different charge properties of the biomarker/probe complex as compared to the uncomplexed components is exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J. Chromatogr B Biomed Sci Appl Oct. 10, 1997; 699(1-2):499-525). Gel electrophoresis can also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically used. Appropriate conditions to the particular assay and components thereof will be well known to one skilled in the art.

In another embodiment, the level of biomarker mRNA is determined both by in situ and by in vitro formats in a biological sample using methods known in the art. Many expression detection methods use isolated RNA. For in vitro methods, any RNA isolation technique that does not select against the isolation of mRNA is utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (U.S. Pat. No. 4,843,155).

In one embodiment, the isolated mRNA is used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe is, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or a genomic DNA encoding a biomarker described herein. Determination of appropriate stringency is identified through routine testing according to conventional molecular techniques. Other suitable probes for use in the diagnostic assays described herein. Hybridization of an mRNA with the probe indicates that a biomarker in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array according to manufacturer's instructions. A skilled artisan can readily adapt known mRNA detection methods for use in detecting the level of mRNA encoded by the biomarkers described herein.

In another embodiment, a method for determining the level of mRNA biomarker in a sample involves the process of nucleic acid amplification, e.g., by reverse transcriptase-polymerase chain reaction (RT-PCR; e.g., the experimental embodiment set forth in Mullis, 1987, U.S. Pat. No. 4,683,202), ligase chain reaction (e.g., Barany, 1991, Proc. Natl. Acad. Sci. USA, 88:189-193), self sustained sequence replication (e.g., Guatelli et al., 1990, Proc. Natl. Acad. Sci. USA 87:1874-1878), transcriptional amplification system (e.g., Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the tumor cells prior to detection. In such methods, a cell or tissue sample is prepared/processed using known histological methods. The sample is then immobilized on a support, typically a glass slide, and then contacted with a probe that can hybridize to mRNA that encodes the biomarker.

In another embodiment, determinations is based on the normalized expression level of the biomarker. Expression levels are normalized by correcting the absolute expression level of a biomarker by comparing its expression to the expression of a gene that is not a biomarker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-tumor sample, or between samples from different sources.

In another embodiment, the expression level is provided as a relative expression level. In one non-limiting example of a method to determine a relative expression level of a biomarker (e.g., DUSP1), the level of expression of the biomarker is determined for 10 or more, 20 or more, 30 or more, 40 or more, or 50 or more samples of normal versus cancer cell isolates prior to the determination of the expression level for the sample in question. The mean expression level of each of the genes or proteins assayed in the larger number of samples is determined and this is used as a baseline expression level for the biomarker. The expression level of the biomarker determined for the test sample (absolute level of expression) is then divided by the mean expression value obtained for that biomarker. This provides a relative expression level.

In another embodiment, a biomarker protein is detected. One type of agent for detecting biomarker protein is an antibody capable of binding to such a protein or a fragment thereof such as, for example, a detectably labeled antibody. Antibodies is polyclonal or monoclonal. An intact antibody, or an antigen binding fragment thereof (e.g., Fab, F(ab′)2, Fv, scFv, single binding chain polypeptide) is used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it is detected with fluorescently labeled streptavidin.

In one embodiment, proteins from tumor cells is isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of methods is employed to determine whether a sample contains a protein that binds to a given antibody. These methods include, but are not limited to, enzyme immunoassay (EIA), radioimmunoassay (RIA), Western blot analysis, immunohistochemistry and enzyme linked immunoabsorbant assay (ELISA). A skilled artisan can readily adapt known protein/antibody detection methods for use in determining whether tumor cells express a biomarker.

In one embodiment, antibodies, or antibody fragments or derivatives, is used in methods such as Western blots or immunofluorescence techniques to detect the expressed proteins. In such uses, either the antibody or proteins is immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. One will know, or can determine, other suitable carriers for binding antibody or antigen, and will be able to adapt such support for use in the present methods. For example, proteins isolated from tumor cells is run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

For ELISA assays, specific binding pairs is of the immune or non-immune type. Immune specific binding pairs are exemplified by antigen-antibody systems or hapten/anti-hapten systems. There is mentioned fluorescein/anti-fluorescein, dinitrophenyl/anti-dinitrophenyl, biotin/anti-biotin, peptide/anti-peptide and the like. The antibody member of the specific binding pair is produced by customary methods familiar to those skilled in the art. Such methods involve immunizing an animal with the antigen member of the specific binding pair. If the antigen member of the specific binding pair is not immunogenic, e.g., a hapten, it is covalently coupled to a carrier protein to render it immunogenic. Non-immune binding pairs include systems wherein the two components share a natural affinity for each other but are not antibodies. Exemplary non-immune pairs are biotin-streptavidin, intrinsic factor-vitamin B12, folic acid-folate binding protein and the like.

A variety of methods are available to covalently label antibodies with members of specific binding pairs. Methods are selected based upon the nature of the member of the specific binding pair, the type of linkage desired, and the tolerance of the antibody to various conjugation chemistries. Biotin is covalently coupled to antibodies by utilizing commercially available active derivatives. Some of these are biotin-N-hydroxy-succinimide which binds to amine groups on proteins; biotin hydrazide which binds to carbohydrate moieties, aldehydes and carboxyl groups via a carbodiimide coupling; and biotin maleimide and iodoacetyl biotin which bind to sulfhydryl groups. Fluorescein is coupled to protein amine groups using fluorescein isothiocyanate. Dinitrophenyl groups is coupled to protein amine groups using 2,4-dinitrobenzene sulfate or 2,4-dinitrofluorobenzene. Other standard methods of conjugation is employed to couple monoclonal antibodies to a member of a specific binding pair including dialdehyde, carbodiimide coupling, homofunctional cross-linking, and heterobifunctional cross-linking. Carbodiimide coupling is an effective method of coupling carboxyl groups on one substance to amine groups on another. Carbodiimide coupling is facilitated by using the commercially available reagent 1-ethyl-3-(dimethyl-aminopropyl)-carbodiimide (EDAC).

Homobifunctional cross-linkers, including the bifunctional imidoesters and bifunctional N-hydroxysuccinimide esters, are commercially available and are employed for coupling amine groups on one substance to amine groups on another. Heterobifunctional cross-linkers are reagents which possess different functional groups. The most common commercially available heterobifunctional cross-linkers have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups is a photoactive aryl nitrene, which upon irradiation reacts with a variety of groups.

A detectably-labeled antibody or detectably-labeled member of the specific binding pair is prepared via coupling to a reporter, which is a radioactive isotope, enzyme, fluorogenic, chemiluminescent or electrochemical materials. Two commonly used radioactive isotopes are 125I and 3H. Standard radioactive isotopic labeling procedures include the chloramine T, lactoperoxidase and Bolton-Hunter methods for ¹²⁵I and reductive methylation for ³H. The term “detectably-labeled” refers to a molecule labeled in such a way that it is readily detected by the intrinsic enzymatic activity of the label or by the binding to the label of another component, which can itself be readily detected.

Enzymes suitable for use in this method include, but are not limited to, horseradish peroxidase, alkaline phosphatase, β-galactosidase, glucose oxidase, luciferases, including firefly and renilla, β-lactamase, urease, green fluorescent protein (GFP) and lysozyme. Enzyme labeling is facilitated by using dialdehyde, carbodiimide coupling, homobifunctional crosslinkers and heterobifunctional crosslinkers as described above for coupling an antibody with a member of a specific binding pair.

The labeling method depends on the functional groups available on the enzyme and the material to be labeled, and the tolerance of both to the conjugation conditions. The labeling method used may be one of, but not limited to, any conventional methods currently employed including those described by Engvall and Pearlmann, Immunochemistry 8, 871 (1971), Avrameas and Temynck, Immunochemistry 8, 1175 (1975), Ishikawa et al., J. Immunoassay 4(3):209-327 (1983) and Jablonski, Anal. Biochem. 148:199 (1985).

Labeling is accomplished by indirect methods such as using spacers or other members of specific binding pairs. An example of this is the detection of a biotinylated antibody with unlabeled streptavidin and biotinylated enzyme, with streptavidin and biotinylated enzyme being added either sequentially or simultaneously. Thus, an antibody used to detect may be detectably-labeled directly with a reporter or indirectly with a first member of a specific binding pair. When the antibody is coupled to a first member of a specific binding pair, then detection is effected by reacting the antibody-first member of a specific binding complex with the second member of the binding pair that is labeled or unlabeled as mentioned above.

The unlabeled detector antibody is detected by reacting the unlabeled antibody with a labeled antibody specific for the unlabeled antibody. In this instance “detectably-labeled” as used above is taken to mean containing an epitope by which an antibody specific for the unlabeled antibody can bind. Such an anti-antibody is labeled directly or indirectly using any of the approaches discussed above. For example, the anti-antibody is coupled to biotin which is detected by reacting with the streptavidin-horseradish peroxidase system discussed above. Thus, in one embodiment, biotin is utilized. The biotinylated antibody is in turn reacted with streptavidin-horseradish peroxidase complex. Orthophenylenediamine, 4-chloro-naphthol, tetramethylbenzidine (TMB), ABTS, BTS or ASA is used for chromogenic detection.

In one immunoassay format, a forward sandwich assay is used in which the capture reagent has been immobilized using conventional techniques on the surface of a support. Suitable supports used in assays include, but limited to, synthetic polymer supports, such as polypropylene, polystyrene, substituted polystyrene, e.g., aminated or carboxylated polystyrene, polyacrylamides, polyamides, polyvinylchloride, glass beads, agarose, or nitrocellulose.

A combination of two or more of the assays described above can also be used to assess one or more biomarkers.

The values obtained from the test and/or control samples is statistically processed using any suitable method of statistical analysis to establish a suitable baseline level using standard methods in the art for establishing such values. Statistical significance is readily determined as further described, for example, in U.S. patent application Ser. No. 11/781,946. In one embodiment, a statistical significance is at least p<0.05.

Treatment of Skin Cancer

The present application relates generally to methods of treatment of skin cancer using DUSP1 as described herein. In one embodiment, it relates to the use of DUSP1 or an active fragment thereof, a nucleic acid that encodes a protein comprising DUSP1 or an active fragment thereof, and analog, or a prodrug thereof in treating a skin cancer.

In one embodiment, provided is a method for treating a patient suffering from a skin cancer by administering an effective amount of DUSP1 or a fragment thereof as described herein.

In another embodiment, provided is a method for treating a patient suffering from a skin cancer by administering an effective amount of DUSP1 or a fragment thereof as described herein in combination with one or more additional active ingredients (e.g., anticancer agents) and/or treatment regimens (e.g., surgery).

In one embodiment, soluble fragments of DUSP1 is used in vitro to determine the effect on melanoma cell lines. In another embodiment, one or more agents that modulate DUSP1 expression are administered to a subject such as a mammal (e.g., a human), suffering from a medical disorder, e.g., a skin cancer. In one embodiment, the skin cancer is melanoma.

Examples of agents useful in enhancing DUSP1 activity (e.g., expression level, protein level or protein activity) include, but are not limited to, angiotensin (e.g., angiotensin-(1-7), angiotensin II (Tallant et al., Hypertension 50: e75-e155, 2007), aldosterone, AG1478 (Min et al., Circ Res 97(5):434-42, 2005), and U0126 (Hotokezaka et al., J. Biol. Chem. 277(49): 47366-47372, 2002).

In one embodiment, provided herein are methods for treating skin cancer in a subject that has developed resistance to a skin cancer therapy comprising administering an effective amount of a compound comprising DUSP1.

Skin cancers, including melanoma, is assayed for growth, metastasis, and response to treatment via multiple known standards. One would understand that classification and staging systems described herein represent one means to assess treatment of cancers, e.g., skin cancer and/or melanoma, described herein; additionally, other staging schemes are known in the art and may be used in connection with the methods described herein. By way of example only, the TNM classification of malignant tumors may be used as a cancer staging system to describe the extent of cancer in a patient's body. T describes the size of the tumor and whether it has invaded nearby tissue, N describes regional lymph nodes that are involved, and M describes distant metastasis. TNM is maintained by the International Union Against Cancer (UICC) and is used by the American Joint Committee on Cancer (AJCC) and the International Federation of Gynecology and Obstetrics (FIGO). One would understand that not all tumors have TNM classifications such as, for example, brain tumors. Generally, T (0, 1-4) is measured as the size or direct extent of the primary tumor. N (0-3) refers to the degree of spread to regional lymph nodes: N0 means that tumor cells are absent from regional lymph nodes, N1 means that tumor cells spread to the closest or small numbers of regional lymph nodes, N2 means that tumor cells spread to an extent between N1 and N3; N3 means that tumor cells spread to most distant or numerous regional lymph nodes. M (0/1) refers to the presence of metastasis: M0 means that no distant metastasis are present; M1 means that metastasis has occurred to distant organs (beyond regional lymph nodes). Other parameters may also be assessed. G (1-4) refers to the grade of cancer cells (i.e., they are low grade if they appear similar to normal cells, and high grade if they appear poorly differentiated). R (0/1/2) refers to the completeness of an operation (i.e., resection-boundaries free of cancer cells or not). L (0/1) refers to invasion into lymphatic vessels. V (0/1) refers to invasion into vein. C (1-4) refers to a modifier of the certainty (quality) of V.

In one embodiment, provided herein are methods for degrading or inhibiting the growth of or killing cancer cells comprising contacting the cells with an amount of a compound described herein effective to degrade, inhibit the growth of or kill cancer cells.

In one embodiment, provided herein are methods of inhibiting tumor size increase, reducing the size of a tumor, reducing tumor proliferation or preventing tumor proliferation in an individual comprising administering to said individual an effective amount of a compound described herein to inhibit tumor size increase, reduce the size of a tumor, reduce tumor proliferation or prevent tumor proliferation. Treatment of tumors in some cases includes stasis of symptoms, that is, by treating the patient, the cancer does not worsen and survival of the patient is prolonged.

Patients may be assessed with respect to symptoms at one or more multiple time points including prior to, during, and after treatment regimens. Treatment can result in improving the subject's condition and is assessed by determining if one or more of the following events has occurred: decreased tumor size, decreased tumor cell proliferation, decreased numbers of cells, decreased neovascularization and/or increased apoptosis. One or more of these occurrences may, in some cases, result in partial or total elimination of the cancer and prolongation of survival of the patient. Alternatively, for terminal stage cancers, treatment may result in stasis of disease, better quality of life and/or prolongation of survival. Other methods of assessing treatment are known in the art and contemplated herein.

Primary outcome measures may be assessed for patients treated using the methods described herein and include, for example, progression-free survival. In one embodiment, an increase in progression free survival is observed in an amount of by about 2-fold, 5-fold, 10-fold, 20-fold, 50-fold or more compared to lack of treatment. In another embodiment, progression free survival increases by about 3 months, about 6 months, about 9 months, about 12 months, about 18 months, about 2 years, about 3 years, about 4 years, about 5 years or more compared to lack of treatment.

Secondary outcome measures may also be assessed and include duration of response, time to tumor progression, overall survival, serious and non-serious adverse events. For example, a treatment may prevent progression of the disease (i.e., stasis) or may result in an improvement. Alternately, or in addition, other goals is measured with respect to one or more of the following: decreased tumor burden, decreased neovascularization, reduced side effects, decreased adverse reactions, and/or increased patient compliance.

Nucleic Acids, Vectors, and Host Cells

The human DUSP1 gene contains four exons and three introns coding for an inducible mRNA that is approximately 2.4 kilobases long. The promoter/enhancer region of this gene contains multiple AP-2, trans-acting transcription factor 1, and camp responsive element sites but only one site for AP-1, neurofibromin 1, and TATA box. Other binding motifs, such as an E box and three GC boxes, are localized between positions −110 and −30. A possible binding site for p53 protein is found in the second intron. The DUSP1 protein is approximately 367 amino acids long and it contains multiple domains. The C-terminal portion (amino acid residues 225-367) of DUSP1 contains the catalytic active site that performs the phosphatase function (amino acid residues 256-264) as well as the region which negatively regulates the phosphatase activity (amino acid residues 315-367). The N-terminal portion (amino acid residues 1-150) contains the nuclear targeting sequence (amino acid residues 13-17), the kinase binding domain (amino acid residues 53-55), and cdc25 homology domains A (amino acid residues 26-46) and B (amino acid residues 116-143). Fragments and/or truncated forms of DUSP1 that retain desired activity (e.g., phosphotase activity) is constructed and expressed. Assays and models to test the activity of DUSP1 proteins, fragment, and/or truncated forms thereof are known in the art and contemplated herein.

In one embodiment, provided are vectors containing a polynucleotide (nucleic acid, DNA) encoding DUSP1 protein or fragment thereof and expression of DUSP1 proteins or fragments thereof.

The expression of proteins in prokaryotic cells, such as E. coli, is well established in the art. Expression in eukaryotic cells in culture is also available to those skilled in the art. A wide variety of unicellular host cells are also useful in expressing the DNA sequences. These hosts include, but not limited to, eukaryotic and prokaryotic hosts, such as strains of E. coli, Pseudomonas, Bacillus, Streptomyces, fungi such as yeasts, and animal cells, such as CHO, YB/20, NSO, SP2/0, R1.1, B-W and L-M cells, African Green Monkey kidney cells (e.g., COS1, COS 7, BSC1, BSC40, and BMT10), insect cells (e.g., Sf9), and human cells and plant cells in tissue culture.

One skilled in the art will be able to select the proper vectors, expression control sequences, and hosts without undue experimentation to accomplish the desired expression without departing from the scope of this application. For example, in selecting a vector, the host must be considered because the vector must function in it. The vector's copy number, the ability to control that copy number, and the expression of any other proteins encoded by the vector, such as antibiotic markers, will also be considered. One of ordinary skill in the art can select the proper vectors, expression control sequences, and hosts to accomplish the desired expression without departing from the scope of this application. For example, in selecting a vector, the host is considered because the vector functions in it. The vector's copy number, the ability to control that copy number, and the expression of any other proteins encoded by the vector, such as antibiotic markers, can also be considered.

The nucleotide and polypeptide sequences for various genes have been previously disclosed, and is found at computerized databases known to those of ordinary skill in the art. One such database is the National Center for Biotechnology Information's Genbank and GenPept databases. The coding regions for these known genes is amplified and/or expressed using the techniques disclosed herein or by any technique that would be know to those of ordinary skill in the art. Additionally, polypeptide sequences is synthesized by methods known to those of ordinary skill in the art, such as polypeptide synthesis using automated polypeptide synthesis machines, such as those available from Applied Biosystems (Foster City, Calif.).

In one embodiment, useful vectors are contemplated to be those vectors in which the coding portion of the DNA segment, whether encoding a full length protein, polypeptide or smaller peptide, is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid coding for the gene product to control RNA polymerase initiation and expression of the gene.

In one embodiment, the promoter is in the form of the promoter that is naturally associated with a gene, as is obtained by isolating the 5′ non-coding sequences located upstream of the coding segment or exon, for example, using recombinant cloning and/or PCR technology, in connection with the compositions disclosed herein.

In other embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a gene in its natural environment. Such promoters can include promoters normally associated with other genes, and/or promoters isolated from any other bacterial, viral, eukaryotic, or mammalian cell, and/or promoters made by the hand of man that are not “naturally occurring,” that is, containing difference elements from different promoters, or mutations that increase, decrease or alter expression.

Promoters that effectively direct the expression of the DNA segment in the cell type, organism, or even animal, are chosen for expression. The use of promoter and cell type combinations for protein expression is generally known to those of skill in the art of molecular biology, for example, see Sambrook et al., (1989), incorporated herein by reference. The promoters employed is constitutive, or inducible, and is used under the appropriate conditions to direct high level expression of the introduced DNA segment, such as is advantageous in the large-scale production of recombinant proteins or peptides.

At least one module in a promoter generally functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 base pairs (bp) upstream of the start site, although a number of promoters have been shown to contain functional elements downstream of the start site as well. The spacing between promoter elements frequently is flexible, so that promoter function is preserved when elements are inverted or moved relative to one another. In the tk promoter, the spacing between promoter elements is increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In expression, one will typically include a polyadenylation signal to effect proper polyadenylation of the transcript. The nature of the polyadenylation signal is not believed to be crucial and any such sequence may be employed. Polyadenylation signals include, but are not limited to the SV40 polyadenylation signal and the bovine growth hormone polyadenylation signal, convenient and known to function well in various target cells. Also contemplated as an element of the expression cassette is a terminator sequence. These elements can serve to enhance message levels and to minimize read through from the cassette into other sequences.

A specific initiation signal also is required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons is either natural or synthetic. The efficiency of expression may be enhanced by the inclusion of appropriate transcription enhancer elements.

It is contemplated that polypeptides is co-expressed with other selected proteins, wherein the proteins is co-expressed in the same cell or a gene(s) is provided to a cell that already has another selected protein. Co-expression is achieved by co-transfecting the cell with two distinct recombinant vectors, each bearing a copy of either of the respective DNA. Alternatively, a single recombinant vector is constructed to include the coding regions for both of the proteins, which could then be expressed in cells transfected with the single vector. In either event, the term “co-expression” herein refers to the expression of both the gene(s) and the other selected protein in the same recombinant cell.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary (CHO) cells, HeLa cells, baby hamster kidney cells, NSO mouse melanoma cells and many others. A common, bacterial host is, for example, E. coli.

To express a recombinant polypeptide, whether modified or wild-type, as provided herein, one would prepare an expression vector that comprises a wild-type, or modified protein-encoding nucleic acid under the control of one or more promoters. To bring a coding sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded recombinant protein. This is the meaning of “recombinant expression” in this context.

Many standard techniques are available to construct expression vectors containing the appropriate nucleic acids and transcriptional/translational control sequences in order to achieve protein, polypeptide or peptide expression in a variety of host-expression systems. Cell types available for expression include, but are not limited to, bacteria, such as E. coli and B. subtilis transformed with recombinant bacteriophage DNA, plasmid DNA or cosmid DNA expression vectors.

Certain examples of prokaryotic hosts are E. coli strain RR1, E. coli LE392, E. coli B, E. coli X 1776 (ATCC No. 31537) as well as E. coli W3110 (F-, lambda-, prototrophic, ATCC No. 273325); bacilli such as B. subtilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which is used by the microbial organism for expression of its own proteins.

In addition, phage vectors containing replicon and control sequences that are compatible with the host microorganism is used as transforming vectors in connection with these hosts. For example, the phage lambda GEM™-11 may be utilized in making a recombinant phage vector which is used to transform host cells, such as E. coli LE392.

Useful vectors include pIN vectors; and pGEX vectors, for use in generating glutathione S-transferase (GST) soluble fusion proteins for later purification and separation or cleavage. Other suitable fusion proteins are those with β-galactosidase, ubiquitin, and the like.

Promoters that are most commonly used in recombinant DNA construction include the β-lactamase (penicillinase), lactose and tryptophan (tip) promoter systems. While these are the most commonly used, other microbial promoters have been discovered and utilized, and details concerning their nucleotide sequences have been published, enabling those of skill in the art to ligate them functionally with plasmid vectors.

The following details concerning recombinant protein production in bacterial cells, such as E. coli, are provided by way of exemplary information on recombinant protein production in general, the adaptation of which to a particular recombinant expression system will be known to those of skill in the art.

Bacterial cells, for example, E. coli, containing the expression vector, are grown in any of a number of suitable media, for example, LB. The expression of the recombinant protein may be induced, e.g., by adding IPTG to the media or by switching incubation to a higher temperature. After culturing the bacteria for a further period, generally of between 2 and 24 hours (h), the cells are collected by centrifugation and washed to remove residual media.

The bacterial cells are then lysed, for example, by disruption in a cell homogenizer and centrifuged to separate the dense inclusion bodies and cell membranes from the soluble cell components. This centrifugation is performed under conditions whereby the dense inclusion bodies are selectively enriched by incorporation of sugars, such as sucrose, into the buffer and centrifugation at a selective speed.

If the recombinant protein is expressed in the inclusion bodies, as is the case in many instances, these is washed in any of several solutions to remove some of the contaminating host proteins, then solubilized in solutions containing high concentrations of urea (e.g., 8 M) or chaotropic agents such as guanidine hydrochloride in the presence of reducing agents, such as β-mercaptoethanol or DTT (dithiothreitol).

It is contemplated that the polypeptides produced by the methods described herein is overexpressed, i.e., expressed in increased levels relative to its natural expression in cells. Such overexpression is assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific polypeptides in relation to the other proteins produced by the host cell and, e.g., visible on a gel.

Expression vectors provided herein comprise a polynucleotide encoding DUSP1 protein or fragment thereof in a form suitable for expression of the polynucleotide in a host cell. The expression vectors generally have one or more regulatory sequences, selected on the basis of the host cells to be used for expression, which is operatively linked to the polynucleotide sequence to be expressed. It will be appreciated by those skilled in the art that the design of the expression vector can depend on such factors as the choice of the host cell to be transformed, the level of expression of protein desired, and the like. The expression vectors described herein is introduced into host cells to produce proteins, including fusion proteins, encoded by polynucleotides as described herein (e.g., DUSP1 protein or fragment thereof, fusion protein, and the like). In addition, DUSP1 or a fragment thereof, or a fusion protein thereof is expressed in bacterial cells such as E. coli. Alternatively, the expression vector is transcribed and translated in vitro.

In one embodiment, provided gene delivery vehicles for the delivery of polynucleotides to cells, tissue, or a mammal for expression. For example, a polynucleotide sequence provided herein is administered either locally or systemically in a gene delivery vehicle. These constructs can utilize viral or non-viral vector approaches in in vivo or ex vivo modality. Expression of such coding sequences is induced using endogenous mammalian or heterologous promoters. Expression of the coding sequence in vivo is either constitutive or regulated. Provided herein are gene delivery vehicles capable of expressing the contemplated polynucleotides including viral vectors.

In another embodiment, provided are purified and substantially purified polypeptides expressed using one or more of the methods described herein.

Where the term “substantially purified” is used, this will refer to a composition in which the specific polypeptide forms the major component of the composition, such as constituting about 50% of the proteins in the composition or more. In one embodiment, a substantially purified polypeptide will constitute more than about 60%, about 70%, about 80%, about 90%, about 95%, about 99% or even more of the polypeptides in the composition.

Various methods for quantifying the degree of purification of polypeptides will be known to those of skill in the art in light of the present disclosure. These include, but not limited to, determining the specific protein activity of a fraction, or assessing the number of polypeptides within a fraction by gel electrophoresis.

In one embodiment, to purify a desired polypeptide a natural or recombinant composition comprising at least some specific polypeptides will be subjected to fractionation to remove various other components from the composition. In addition to those techniques described in detail herein below, various other techniques suitable for use in protein purification will be well known to those of skill in the art. These include, for example, precipitation with ammonium sulfate, PEG, antibodies and the like or by heat denaturation, followed by centrifugation, chromatography steps such as ion exchange, gel filtration, reverse phase, hydroxylapatite, lectin affinity and other affinity chromatography steps, isoelectric focusing, gel electrophoresis, and combinations of such and other techniques.

In another embodiment, the purification of a specific fusion protein is made using a specific binding partner. Such purification methods are routine in the art and any fusion protein purification method is practiced. This is exemplified by the generation of a specific protein-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography. However, given many DNA and proteins are known, or may be identified and amplified using the methods described herein, any purification method can now be employed.

There is no general requirement that the polypeptides always be provided in their most purified state. Indeed, it is contemplated that less substantially purified polypeptides which are nonetheless enriched in the desired protein compositions, relative to the natural state, will have utility in certain embodiments. Polypeptides exhibiting a lower degree of relative purification may have advantages in total recovery of protein product, or in maintaining the activity of an expressed protein.

Compositions

Each of the compounds described herein is used as a composition when combined with an acceptable carrier or excipient. Such compositions are useful for in vitro analysis or for administration to a subject in vivo or ex vivo for treating a subject with the disclosed compounds.

The pharmaceutical compositions comprise, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material depends on the route of administration.

In one embodiment, pharmaceutical formulations comprising a protein of interest identified by the methods described herein is prepared for storage by mixing the protein having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In another embodiment, the formulation described herein can also contain more than one active compound as necessary for the particular indication being treated. Such molecules are suitably present in combination in amounts that are effective for the intended purpose.

Acceptable carriers are physiologically acceptable to a patient and retain the therapeutic properties of the compounds with which they are administered. Acceptable carriers and their formulations are and generally described in, for example, Remington' pharmaceutical Sciences (18th Edition, ed. A. Gennaro, Mack Publishing Co., Easton, Pa. 1990). One exemplary carrier is physiological saline. Exemplary carriers and excipients have been provided elsewhere herein.

In one embodiment, provided herein are pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. The pharmaceutical compositions and formulations include an amount of a compound described herein, for example, an effective amount of modified fusion protein described herein, and a pharmaceutically or physiologically acceptable carrier.

In one embodiment, compositions is formulated to be compatible with a particular route of administration, systemic or local. Thus, compositions include carriers, diluents, or excipients suitable for administration by various routes. Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

In another embodiment, the compositions can further comprise, if needed, an acceptable additive in order to improve the stability of the compounds in composition and/or to control the release rate of the composition. Acceptable additives do not alter the specific activity of the subject compounds. Exemplary acceptable additives include, but are not limited to, a sugar such as mannitol, sorbitol, glucose, xylitol, trehalose, sorbose, sucrose, galactose, dextran, dextrose, fructose, lactose and mixtures thereof. Acceptable additives is combined with acceptable carriers and/or excipients such as dextrose. Alternatively, exemplary acceptable additives include, but are not limited to, a surfactant such as polysorbate 20 or polysorbate 80 to increase stability of the peptide and decrease gelling of the solution. The surfactant is added to the composition in an amount of 0.01% to 5% of the solution. Addition of such acceptable additives increases the stability and half-life of the composition in storage.

The pharmaceutical composition is administered subcutaneously, intramuscularly, intraperitoneally, orally or intravenously. Aerosol delivery of the compositions is also contemplated herein using conventional methods. For example, intravenous delivery is now possible by cannula or direct injection or via ultrasound guided fine needle. Mishra (Mishra et al., Expert Opin. Biol., 3(7):1173-1180 (2003)) provides for intratumoral injection.

Formulations for enteral (oral) administration is contained in a tablet (coated or uncoated), capsule (hard or soft), microsphere, emulsion, powder, granule, crystal, suspension, syrup or elixir. Conventional non-toxic solid carriers which include, for example, pharmaceutical grades of mannitol, lactose, starch, magnesium stearate, sodium saccharin, talcum, cellulose, glucose, sucrose, magnesium carbonate, is used to prepare solid formulations. Supplementary active compounds (e.g., preservatives, antibacterial, antiviral and antifungal agents) can also be incorporated into the formulations. A liquid formulation can also be used for enteral administration. The carrier is selected from various oils including petroleum, animal, vegetable or synthetic, for example, peanut oil, soybean oil, mineral oil, sesame oil. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Compositions for injection include aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier is a solvent or dispersion medium containing, for example, water, ethanol, polyol (for example, glycerol, propylene glycol, and liquid polyetheylene glycol, and the like), and suitable mixtures thereof. Fluidity is maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as manitol, sorbitol, and sodium chloride may be included in the composition. The resulting solutions is packaged for use as is, or lyophilized; the lyophilized preparation can later be combined with a sterile solution prior to administration.

Compositions is conventionally administered intravenously, such as by injection of a unit dose, for example. For injection, an active ingredient is in the form of a parenterally acceptable aqueous solution which is substantially pyrogen-free and has suitable pH, isotonicity and stability. One can prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

In one embodiment, the composition is lyophilized. When the compositions are considered for medicaments, or use in any of the methods provided herein, it is contemplated that the composition is substantially free of pyrogens such that the composition will not cause an inflammatory reaction or an unsafe allergic reaction.

Acceptable carriers can contain a compound that stabilizes, increases or delays absorption or clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound is complexed with a composition to render it resistant to acidic and enzymatic hydrolysis, or the compound is complexed in an appropriately resistant carrier such as a liposome. Means of protecting compounds from digestion are known in the art (see, e.g., Fix (1996) Pharm Res. 13:1760 1764; Samanen (1996) J. Pharm. Pharmacol. 48:119 135; and U.S. Pat. No. 5,391,377, describing lipid compositions for oral delivery of therapeutic agents).

For intravenous injection or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as needed.

The compositions is administered in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

A physician or veterinarian can readily determine and prescribe the “effective amount” of the composition required. For example, the physician or veterinarian could start doses of the compounds employed in the composition at levels lower than that required in order to achieve the desired therapeutic effect and gradually increase the dosage until the desired effect is achieved. In one example, the amount of a DUSP1 necessary to bring about prevention and/or therapeutic treatment of the disease is not fixed per se. The amount of DUSP1 administered may vary with the type of disease, extent of the disease, and size of species of the mammal suffering from the disease. Generally, amounts will be in the range of those used for other cytotoxic agents used in the treatment of cancer.

In certain embodiments, currently available techniques, for example (cannulae or convection enhanced delivery, selective release) that attempt to deliver enhanced locally elevated DUSP1 amounts to specific sites may also be desired.

One embodiment contemplates the use of the compositions described herein to make a medicament for treating a condition, disease or disorder described herein. Medicaments is formulated based on the physical characteristics of the patient/subject needing treatment, and is formulated in single or multiple formulations based on the stage of the condition, disease or disorder. Medicaments is packaged in a suitable package with appropriate labels for the distribution to hospitals and clinics wherein the label is for the indication of treating a subject having a disease described herein. Medicaments is packaged as a single or multiple units. Instructions for the dosage and administration of the compositions is included with the packages as described below.

In one embodiment, provided are pharmaceutical compositions comprising a modified toxin or fusion protein thereof described hereinabove and a pharmaceutically acceptable carrier.

Sterile injectable solutions is prepared by incorporating an active ingredient in the required amount in an appropriate solvent with one or a combination of ingredients enumerated above, as required, followed by filtered sterilization. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation are vacuum drying and freeze drying which yields a powder of the active ingredient plus any additional desired ingredient from a previously sterile-filtered solution thereof.

In certain embodiments, provided are methods for further purification of this mixture to obtain preparations essentially comprising fusion proteins. This purification is accomplished by further chromatographic separation which is accomplished by affinity chromatography, for example, using a salt gradient to elute the various species of immunotoxins and gel filtration to separate the immunotoxins from larger molecules.

A gel to be used in purification of compounds described herein is a three dimensional network which has a random structure. Molecular sieve gels are those cross-linked polymers that do not bind or react with the material being analyzed or separated. For gel filtration purposes, a gel material is generally uncharged. The space within the gel is filled with liquid and the liquid phase constitutes the majority of the gel volume. Materials commonly used in gel filtration columns include dextran, agarose and polyacrylamide.

Dextran is commercially available under the name SEPHADEX (Phamacia Fine Chemicals, Inc.). The beads are prepared with various degrees of cross-linking in order to separate different sized molecules by providing various pore sizes. Alkyl dextran is cross-linked with N,N′-methylenebisacrylamide to form SEPHACRYL-S100 to S1000 which allows strong beads to be made that fractionate in larger ranges than SEPHADEX can achieve.

Polyacrylamide is available in a variety of pore sizes to be used for separation of different size particles (Bio-Rad Laboratories, USA). The beads are available in various degrees of fineness to be used in different applications. The coarser the bead, the faster the flow and the poorer the resolution. Superfine is used for maximum resolution, but the flow is very slow. Fine is used for preparative work in large columns which require a faster flow rate. The coarser grades are for large preparations in which resolution is less important than time, or for separation of molecules with a large difference in molecular weights.

In one embodiment, an affinity chromatography method is used where the matrix is a reactive dye-agarose matrix. Blue-SEPHAROSE, a column matrix composed of Cibacron Blue 3GA and agarose or SEPHAROSE is used as the affinity chromatography matrix. Alternatively, SEPHAROSE CL-6B is available as Reactive Blue 2 from Sigma Chemical Company. This matrix binds fusion proteins directly and allows their separation by elution with a salt gradient.

Kits

Further provided herein are kits for the performance of the methods described herein. In one embodiment, such kits or packages include reagents for the detection of DUSP1 expression levels. In further embodiments, the kits contain reagents for the detection of DUSP1 expression levels and a compilation containing DUSP1 expression level indices that have been predetermined to correlate with the presence or absence of skin cancer. In still further embodiments, that compilation contains DUSP1 expression level indices that have been predetermined to correlate with the survivability for a cancer.

In some embodiments, a kit comprises reagents for the detection of DNA, RNA or protein expression levels in a sample of tumor cells from a patient to be treated. Packages and kits can further include one or more components for an assay, such as, for example, an ELISA assay, immunohistochemistry assay, nucleic hybridization assay, etc. Samples to be tested in this application include, for example, blood, plasma, and tissue sections and secretions, urine, lymph, and products thereof, and Packages and kits can further include one or more components for collection of a sample (e.g., a syringe, a cup, a swab, etc.). Packages or kits provided herein can further include any of the other moieties or reagents necessary for the methods provided herein such as, for example, one or more reporter molecules and/or one or more detectable moieties/agents.

Packages and kits can further include a label specifying, for example, a product description, mode of administration and/or indication of treatment. Packages provided herein can include any of the reagents necessary as described herein for treatment of any of the indications described herein. The label or packaging insert can include appropriate written instructions. Kits, therefore, can additionally include labels or instructions for using the kit components in any method described herein. A kit can include a compound in a pack, or dispenser together with instructions for administering the compound in a method described herein. The instructions may be on “printed matter,” e.g., on paper or cardboard within or affixed to the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, magnetic tape, electrical storage media such as RAM and ROM, IC tip and hybrids of these such as magnetic/optical storage media.

### EXAMPLES

### Example 1

### DUSP1 Expression Analysis for Melanoma

A cohort of 61 metastatic melanoma samples with known patient outcomes were used to examine the relationship of DUSP1 presence and overall survival. Sections of the tissues were cut, mounted on a microscope slide, and then analyzed using immunohistochemistry staining methods (Histotechnology: A Self-Instructional Text by Freida L. Carson; Publisher: American Society Clinical Pathology; 2 edition (May 1, 1997) ISBN-10: 089189411X ISBN-13: 978-0891894117).

Tissue Dissection, Fixation, Embedding and Sectioning

The melanoma was excised and subjected to formalin fixation and embedded in paraffin. The formalin fixed paraffin embedded (FFPE) tissue was sectioned at a thickness of 4 microns and sections were placed onto positively charged glass slides. The slide was stained with the designated primary antibody which reacted with the tissue antigen as chosen by the pathologist. A labeled secondary antibody reacted with the primary antibody and coupled to a streptavidin-horseradish peroxidase. This complex reacted with a chromogen to produce a colored stain. The stained slides were viewed by a pathologist under a light microscope. The pathologist performed a semi-quantitative interpretation of the intensity of the staining. Typically, a 0 to 3 scale is utilized with 0 representing no staining or negative result. The pathologist then estimated the proportion of the tumor cells that were stained positively. Typically, a 0 to 100% scale is utilized.

Staining Procedure

The following is a method of detecting an elevated level of DUSP1 in a biological tissue comprising the steps of:

a) providing a slide having a fixed sample contained thereon;

b) deparaffinizing and rehydrating or further processing the sample for histochemistry;

c) rinsing the sample at least once with a buffer or another aqueous liquid;

d) performing any cell conditioning or antigen retrieval for any amount of time

e) rinsing the sample at least once with a buffer or another aqueous liquid; covering the sample with a peroxide or other oxidative agent for any amount of time;

g) rinsing the tissue at least once with a buffer or another aqueous liquid;

h) covering the sample with at least one primary DUSP1 antibody diluted in a Tris and/or phosphate buffered saline (PBS) based diluent, a negative control reagent, or another suitable carrier solution for any amount of time with or without prior or subsequent application of a blocking agent to the sample;

i) rinsing the sample at least once with a buffer or another aqueous liquid;

j) covering the sample at least once with a secondary detection or polymer or multimer reagent for any amount of time;

k) rinsing the sample at least once with a buffer or another aqueous liquid;

l) covering the sample with an alkaline phosphatase or horseradish peroxidase conjugate, an enzymatic agent, or other catalytic agent for any amount of time;

m) rinsing the sample at least once with buffer or another aqueous liquid;

n) covering the sample with any type of enhancing reagent for any amount of time;

o) covering the sample with Fast Red/Naphthol or 3,3′-diaminobenzidine containing solution or another chromagen containing solution for any amount of time;

p) rinsing the sample at least once with water or another aqueous liquid;

q) covering the sample with a Fast Red or 3,3′-diaminobenzidine containing solution or another chromagen containing solution for any amount of time;

r) rinsing the sample at least once with water or another aqueous liquid;

s) covering the sample with a hematoxylin and/or bluing reagent counterstain or other counterstain reagent for any amount of time;

t) rinsing the sample at least once with a buffer or another aqueous liquid;

u) repeatedly dipping the sample and the slide in distilled water until the slide is clear;

v) dehydrating the sample;

w) applying a cover slip over the sample contained on the slide; and

x) detecting an elevated level of MKP-1/DUSP1 by examining the sample under a microscope and comparing the sample to a control.

An automated or manual staining procedure was performed and all slides stained on the Ventana Benchmark XT Autostainer (Ventana Medical Systems, Tucson, Ariz.), catalog #N750-BMKLT-M, with the following protocol. Approximately 100 μL of each reagent required below is applied to each slide.

Slides containing 4 microns of formalin-fixed paraffin-embedded tissue were placed onto the Ventana Benchmark XT Autostainer and the following steps under the ultraView™ Universal Alkaline Phosphatase RED Detection kit were programmed:

A. Select deparaffinization and rehydration to be performed online by the Ventana Benchmark XT Autostainer;

B. Select standard cell conditioning using CC1 (Ventana Medical Systems, Tucson, Ariz.), catalog #950-124.

C. Select DUSP1 antibody and incubate at 37° C. for 32 minutes.
1. Antibody: DUSP1 (Santa Cruz Biotechnology, CA), catalog #sc-1199.
   a. Dilution: 1:1000
   b. Lot #: H2007
   c. Clone: Polyclonal (V-15)
   d. Host: Rabbit
2. Diluent: Bond Primary Antibody Diluent (Leica, United Kingdom), catalog #AR9352.

D. Select ultraView™ Universal Alkaline Phosphatase RED (Ventana Medical Systems, Tucson, Ariz.), catalog #760-501, for preprogrammed amount of time as follows.
1. Apply one drop of ultraView™ Universal AP Red Multimer (Ventana Medical Systems, Tucson, Ariz.), catalog #253-4327, for 12 minutes.
2. Apply one drop of ultraView™ Universal AP Red Enhancer (Ventana Medical Systems, Tucson, Ariz.), catalog #253-4326, for 4 minutes.
3. Apply one drop of ultraView™ Universal AP Red Fast Red A (Ventana Medical Systems, Tucson, Ariz.), catalog #253-4329, and one drop of ultraView™ Universal AP Red Naphthol (Ventana Medical Systems, Tucson, Ariz.), catalog #253-4328, simultaneously for 8 minutes.
4. Apply one drop of ultraView™ Universal AP Red Fast Red B (Ventana Medical Systems, Tucson, Ariz.), catalog #253-4330, for 4 minutes.

E. Select counterstain with Hematoxylin (Ventana Medical Systems, Tucson, Ariz.), catalog #760-2021, for 4 minutes.

F. Select post-counterstain with Bluing Reagent (Ventana Medical Systems, Tucson, Ariz.), catalog #760-2037, for 4 minutes.

G. Remove all slides from Ventana Benchmark XT Autostainer upon completion and rinse with soap and warm water.

H. Rinse slides with water until no soap is present.

I. Dehydrate and coverslip slides per routine procedures.

### Experimental Results

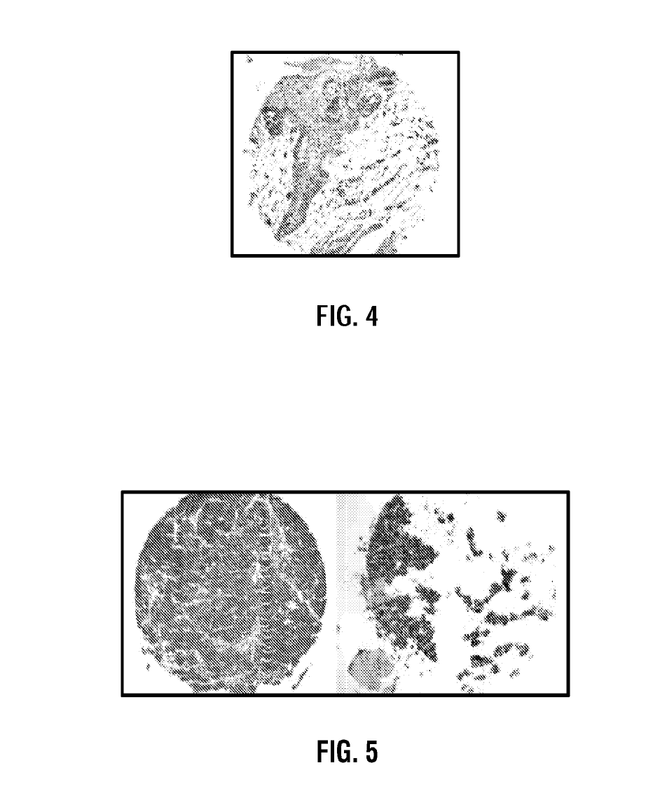

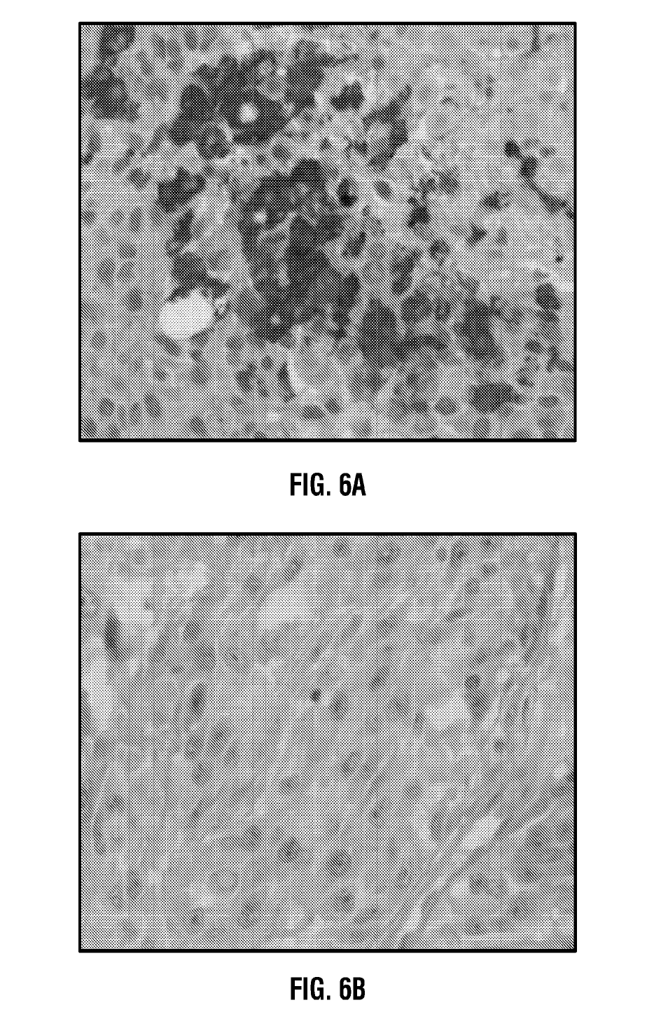

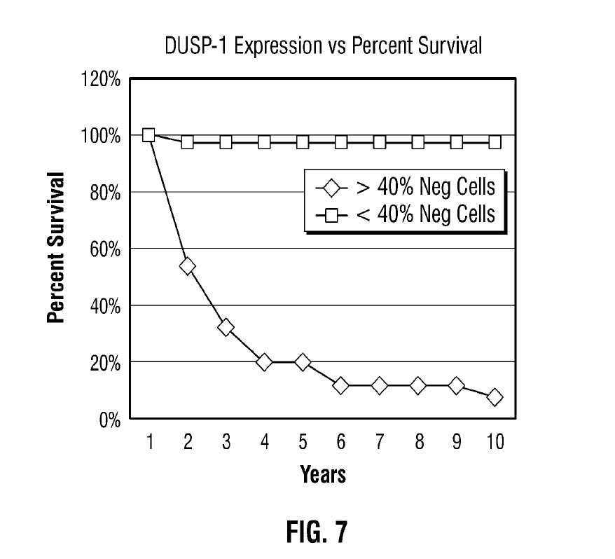

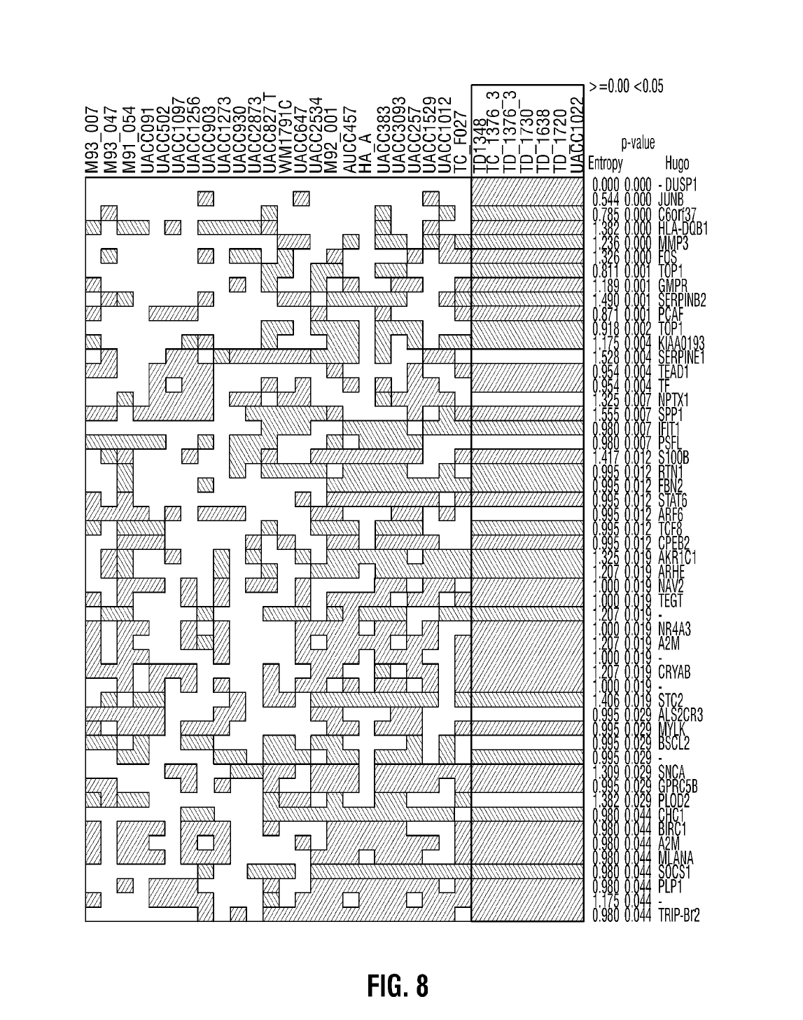

Immunohistochemistry results on melanoma tumors showed that DUSP1 is significantly overexpressed at the leading edge of tumors diagnosed as melanoma. An exemplary immunohistochemistry result for DUSP1 staining at the leading edge of a tumor is shown in FIG. 4. FIG. 5 shows sections of two small tumors from a very long term survivor of melanoma removed at year 15 and year 17 (left to right respectively). This long term survivor had small tumors removed multiple times during 17 years of living with Stage 1V melanoma. The tumor samples showed high levels of DUSP1 expression as evidenced by the intense staining of the DUSP1 in the cells of the cancerous tissue.

Retrospective comparison of survival information was performed using a sample consisting of 61 confirmed cases of melanoma with known outcomes stained for DUSP1 by IHC as described above. The results are shown in Table 1 (below) along with the pathological characteristics of each of the cancers (Breslow and Clark's level and stage).

TABLE 1
Summary of IHC Results of 61 melanoma cases

| Date of | | | | | | | | Disease |
|---|---|---|---|---|---|---|---|---|
| Initial | Age at | | Clark | Breslow | | | Death from | Free |
| Diagnosis | Diagnosis | Sex | Level | Level | | Stage | Melanoma | followup |
| Jun. 28, 1990 | 62 | F | Clark Level V | .9 | cm | 3 | Yes, Mar. 22, 1991 | |
| November 1982 | 71 | M | Clark Level II | | | Localized | | October 1993 |
| Feb. 28, 1994 | 80 | M | Clark Level II | .19 | mm | Localized | | February 2005 |
| Feb. 16, 1995 | 78 | F | Clark Level II | .4 | mm | Localized | | January 2006 |
| Jul. 10, 1995 | 64 | M | Clark Level II | .29 | mm | Localized | | February 2006 |
| Apr. 28, 1994 | 74 | F | Clark Level II | .49 | mm | Localized | | April 2005 |
| Oct. 19, 1994 | 74 | F | Clark Level II | .51 | mm | Localized | | March 2006 |
| August 1993 | 28 | F | Clark Level II | .41 | mm | Localized | | February 2006 |
| Mar. 22, 1985 | 52 | M | Clark Level II | .325 | mm | Localized | | Nov. 24, 2000 |
| November 1987 | 57 | F | Clark Level IV | | | Localized | | August 2002 |
| September 1979 | 27 | F | Clark level II | | | 2 | | November 1995 |
| Oct. 8, 1982 | 63 | M | Clark Level III | .3 | mm | Localized | | Jan. 27, 1998 |
| May 10, 1985 | 68 | F | Clark Level III | 2.4 | mm | 1B | | Jun. 15, 2001 |
| May 1986 | 62 | F | Clark Level III | 1.34 | mm | 2 | | June 2002 |
| Sep. 21, 1990 | 59 | M | Clark Level IV | 1.9 | mm | 2 | | Mar. 20, 2006 |
| Sep. 30, 1984 | 55 | M | Clark Level II | 0.64 | mm | 1 | | May 20, 2002 |
| September 1984 | 59 | F | Clark Level III | .9 | mm | 1B | | March 2004 |
| July 1985 | 56 | F | Clark Level III | 1.5 | mm | 2 | | September 2005 |
| Jan. 23, 1986 | 56 | M | Clark Level IV | 2.5 | mm | 2A | | Apr. 21, 2006 |
| Sep. 18, 1985 | 69 | M | Clark Level II | .29 | mm | Localized | | Apr. 25, 2006 |
| June 1983 | 44 | M | Clark Level III | 1.05 | mm | Localized | | March 2004 |
| Mar. 6, 1982 | 44 | M | Clark Level III | 0.1 | mm | Localized | | Jul. 1, 2004 |
| Mar. 14, 1983 | 69 | M | Clark Level III | 2.2 | mm | Localized | | Jan. 12, 2005 |
| Aug. 17, 1982 | 59 | M | Clark Level III | .8 | mm | Localized | | Aug. 24, 2006 |
| Oct. 19, 1994 | 72 | F | Clark Level II | .46 | mm | Localized | | December 2006 |
| Mar. 20, 1987 | 67 | M | Clark Level III | 1.5 | mm | 1B | | Oct. 1, 2005 |
| July 1988 | 56 | M | Clark Level IV | | | 2A | | June 2006 |
| Oct. 1, 1983 | 52 | M | Clark Level IV | 0.9 | | Localized | | Jan. 22, 1998 |
| July 1983 | 63 | F | Clark Level III | 1.1 | mm | Localized | | October 1999 |
| Aug. 5, 1994 | 69 | M | Clark Level II | .33 | mm | Localized | | June 2006 |
| March 1995 | 62 | M | Clark Level III | 1.35 | mm | Localized | | May 2005 |
| Apr. 19, 1994 | 59 | M | Clark Level II | .72 | mm | Localized | | March 2006 |
| May 1979 | 70 | F | Clark Level III | .42 | mm | Localized | | January 1992 |
| Apr. 30, 1982 | 61 | F | Clark Level III | 4.4 | mm | Localized | | Mar. 22, 2001 |
| November 1978 | 75 | F | Clark Level III | | | 1 | | December 1991 |
| May 15, 1993 | 44 | M | Clark Level II | .27 | mm | Localized | | September 2005 |
| Aug. 18, 1990 | 69 | F | Clark Level II | | | 1 | | Jun. 15, 2003 |
| Sep. 23, 1985 | 64 | F | Clark Level IV | 4.12 | mm | 2B | Apr. 5, 1986 | |
| Feb. 1, 1990 | 56 | m | Clark Level V | 1.5 | mm | 3 | Yes, Sep. 22, 1990 | |
| Aug. 31, 1981 | 49 | M | | | | 2a | Yes, Dec. 15, 1982 | |
| May 22, 1984 | 76 | F | Clark Level IV | 2.9 | mm | Localized | Yes, Mar. 1, 1986 | |
| July 1981 | 63 | M | Clark Level IV | | | Localized | March 1984 | |
| June 1979 | 61 | F | Clark Level III | | | Localized | | March 1992 |
| Jan. 28, 1988 | 59 | F | | 2 | mm | 2B | Yes, Jul. 20, 1990 | |
| Jun. 16, 1993 | 23 | M | Clark Level IV | 5 | mm | 3 | Jul. 1, 1994 | |
| Aug. 1, 1985 | 84 | M | Clark Level IV | 0.8 | mm | Localized | Yes, Jan. 11, 1986 | |
| Aug. 1, 1985 | 84 | M | Clark Level IV | 0.8 | mm | Localized | Yes, Jan. 11, 1986 | |
| Mar. 25, 1992 | 40 | M | Clark Level V | 1.6 | cm | 4A | Yes, Jun. 3, 1993 | |
| Jun. 9, 1988 | | M | | | | 2 | yes, Jul. 9, 1990 | |
| Oct. 5, 1978 | 65 | M | Clark Level III | 11 | mm | Localized | Yes, Nov. 10, 1983 | |
| Apr. 5, 1994 | 76 | M | Clark Level V | 6.5 | mmd | 3 | Yes, Dec. 1, 1995 | |
| Mar. 20, 1990 | 69 | M | | 5 | mm | 3 | Yes, Jul. 6, 1991 | |
| Oct. 8, 1991 | 57 | M | Clark Level II | 0.7 | mm | 1 | Yes, Mar. 22, 1993 | |
| Oct. 5, 1978 | 65 | M | Clark Level III | 11 | mm | Localized | Yes, Nov. 10, 1983 | |
| September 1984 | 57 | F | Clark Level IV | 2 | mm | 2A | | July 2001 |
| Dec. 8, 1987 | 35 | m | Clark Level IV | 2 | mm | 2A | | Mar. 15, 2005 |
| July 1981 | 63 | M | Clark Level IV | | | Localized | March 1984 | |
| Oct. 11, 1995 | 78 | M | Clark Level IV | 5 | mm | Localized | May 1, 1998 | |
| January 1979 | 79 | M | Clark Level V | | | 1 | January 1980 | |
| June 1995 | 62 | M | | | | 3 | December 1995 | |
| January 1980 | 71 | M | Clark Level IV | 1.5 | cm | Localized | October 1982 | |

| Date of | | | | % Cells Positive | | | % Cells | |
|---|---|---|---|---|---|---|---|---|
| Initial | Age at | | | Intensity: | | | Negative | Overall |
| Diagnosis | Diagnosis | Sex | Case # | 3+ | 2+ | 1+ | for DUSP1 | Survival |
| Jun. 28, 1990 | 62 | F | 88 | 30 | 70 | 0 | 0 | 1 |
| November 1982 | 71 | M | 49 | 5 | 95 | 0 | 0 | 11 |
| Feb. 28, 1994 | 80 | M | 101 | 20 | 80 | 0 | 0 | 11 |
| Feb. 16, 1995 | 78 | F | 104 | 5 | 95 | 0 | 0 | 11 |
| Jul. 10, 1995 | 64 | M | 108 | 5 | 95 | 0 | 0 | 11 |
| Apr. 28, 1994 | 74 | F | 109 | 20 | 80 | 0 | 0 | 11 |
| Oct. 19, 1994 | 74 | F | 102 | 30 | 70 | 0 | 0 | 12 |
| August 1993 | 28 | F | 114 | 5 | 95 | 0 | 0 | 13 |
| Mar. 22, 1985 | 52 | M | 61 | 10 | 90 | 0 | 0 | 15 |
| November 1987 | 57 | F | 116 | 0 | 2 | 98 | 0 | 15 |
| September 1979 | 27 | F | 36 | 15 | 85 | 0 | 0 | 16 |
| Oct. 8, 1982 | 63 | M | 43 | 5 | 50 | 45 | 0 | 16 |
| May 10, 1985 | 68 | F | 66 | 10 | 70 | 20 | 0 | 16 |
| May 1986 | 62 | F | 70 | 10 | 30 | 60 | 0 | 16 |
| Sep. 21, 1990 | 59 | M | 85 | 20 | 80 | 0 | 0 | 16 |
| Sep. 30, 1984 | 55 | M | 56 | 20 | 70 | 10 | 0 | 18 |
| September 1984 | 59 | F | 59 | 5 | 95 | 0 | 0 | 20 |
| July 1985 | 56 | F | 64 | 10 | 90 | 0 | 0 | 20 |
| Jan. 23, 1986 | 56 | M | 67 | 20 | 40 | 40 | 0 | 20 |
| Sep. 18, 1985 | 69 | M | 63 | 5 | 95 | 0 | 0 | 21 |
| June 1983 | 44 | M | 52 | 40 | 60 | 0 | 0 | 21 |
| Mar. 6, 1982 | 44 | M | 46 | 5 | 95 | 0 | 0 | 22 |
| Mar. 14, 1983 | 69 | M | 51 | 30 | 40 | 30 | 0 | 22 |
| Aug. 17, 1982 | 59 | M | 48 | 5 | 95 | 0 | 0 | 24 |
| Oct. 19, 1994 | 72 | F | 107 | 0 | 30 | 60 | 10 | 12 |
| Mar. 20, 1987 | 67 | M | 73 | 10 | 20 | 60 | 10 | 18 |
| July 1988 | 56 | M | 82 | 30 | 30 | 30 | 10 | 18 |
| Oct. 1, 1983 | 52 | M | 119 | 5 | 80 | 0 | 15 | 15 |
| July 1983 | 63 | F | 53 | 2 | 0 | 80 | 18 | 16 |
| Aug. 5, 1994 | 69 | M | 100 | 40 | 40 | 0 | 20 | 12 |
| March 1995 | 62 | M | 103 | 5 | 70 | 0 | 25 | 10 |
| Apr. 19, 1994 | 59 | M | 106 | 5 | 70 | 0 | 25 | 12 |
| May 1979 | 70 | F | 40 | 5 | 0 | 70 | 25 | 13 |
| Apr. 30, 1982 | 61 | F | 42 | 5 | 20 | 50 | 25 | 19 |
| November 1978 | 75 | F | 35 | 10 | 50 | 10 | 30 | 13 |
| May 15, 1993 | 44 | M | 105 | 5 | 30 | 30 | 35 | 12 |
| Aug. 18, 1990 | 69 | F | 89 | 5 | 30 | 30 | 35 | 13 |
| Sep. 23, 1985 | 64 | F | 57 | 30 | 0 | 30 | 40 | 1 |
| Feb. 1, 1990 | 56 | m | 86 | 20 | 20 | 0 | 60 | 1 |
| Aug. 31, 1981 | 49 | M | 44 | 10 | 20 | 10 | 60 | 1 |
| May 22, 1984 | 76 | F | 58 | 5 | 30 | 0 | 65 | 2 |
| July 1981 | 63 | M | 120 | 5 | 20 | 0 | 75 | 3 |
| June 1979 | 81 | F | 37 | 5 | 0 | 20 | 75 | 13 |
| Jan. 28, 1988 | 59 | F | 78 | 10 | 10 | 0 | 80 | 2 |
| Jun. 16, 1993 | 23 | M | 123 | 5 | 10 | 0 | 85 | 1 |
| Aug. 1, 1985 | 84 | M | 62 | 10 | 0 | 0 | 90 | 1 |
| Aug. 1, 1985 | 84 | M | 122 | 10 | 0 | 0 | 90 | 1 |
| Mar. 25, 1992 | 40 | M | 93 | 5 | 5 | 0 | 90 | 1 |
| Jun. 9, 1988 | | M | 79 | 2 | 5 | 0 | 93 | 2 |
| Oct. 5, 1978 | 65 | M | 47 | 2 | 5 | 0 | 93 | 5 |
| Apr. 5, 1994 | 76 | M | 95 | 0 | 5 | 0 | 95 | 1 |
| Mar. 20, 1990 | 69 | M | 84 | 5 | 0 | 0 | 95 | 1 |
| Oct. 8, 1991 | 57 | M | 90 | 5 | 0 | 0 | 95 | 2 |
| Oct. 5, 1978 | 65 | M | 121 | 0 | 0 | 5 | 95 | 5 |
| September 1984 | 57 | F | 60 | 0 | 0 | 5 | 95 | 9 |
| Dec. 8, 1987 | 35 | m | 72 | 5 | 0 | 0 | 95 | 18 |
| July 1981 | 63 | M | 41 | 2 | 0 | 0 | 98 | 3 |
| Oct. 11, 1995 | 78 | M | 96 | 0 | 2 | 0 | 98 | 3 |
| January 1979 | 79 | M | 115 | 1 | 0 | 0 | 99 | 1 |
| June 1995 | 62 | M | 97 | 1 | 0 | 0 | 99 | 1 |
| January 1980 | 71 | M | 45 | 1 | 0 | 0 | 99 | 2 |

Several examples of the greater utility of the DUSP1 marker are seen in Table 1. Even when the Breslow level was greater than 2 mm, the percent negative cells was a better predictor of long term survival (cases 66, 67 and 42). Likewise, even when the Breslow level was less than 1 mm, the percent negative cells was a greater predictor for overall survival (cases 122, 62 and 90).

The Kaplan-Meier estimator estimates the survival function from life-time data. A plot of the Kaplan-Meier estimate of the survival function is a series of horizontal steps of declining magnitude which, when a large enough sample is taken, approaches the true survival function for that population. The value of the survival function between successive distinct samples observations is assumed to be constant.

As depicted in FIG. 5, Kaplan-Meier plots for all tumors, as well as for tumors segregated on the basis of intensity of DUSP1 immunostaining, clearly showed that increasing levels of DUSP1 protein correlated with increased survival (p<0.001). The more non-DUSP1 expressing tumor cells in the tumor, the shorter the survival time. The percent negative cells in the tumor showed the highest negative correlation with overall survival. The results are shown in Table 2.

TABLE 2
Summary of Data Showing Overall Survival vs. Percent Negative Cells

| | DUSP-1 | | | DUSP-1 | |
|---|---|---|---|---|---|
| | Neg Cells | | | Pos Cells | |
| | (>40% | | | (<40% | |
| OS | Neg Cells) | | OS | Neg Cells) | |
| % remaining alive | year | # alive | % remaining alive | year | # alive |
| 100 | 1 | 24/24 | 100 | 1 | 37/37 |
| 54 | 2 | 13/24 | 97 | 2 | 36/37 |
| 33 | 3 | 8/24 | 97 | 3 | 36/37 |
| 20 | 4 | 5/24 | 97 | 4 | 36/37 |
| 20 | 5 | 5/24 | 97 | 5 | 36/37 |
| 12 | 6 | 3/24 | 97 | 6 | 36/37 |
| 12 | 7 | 3/24 | 97 | 7 | 36/37 |
| 12 | 8 | 3/24 | 97 | 8 | 36/37 |
| 12 | 9 | 3/24 | 97 | 9 | 36/37 |
| 8 | 10 | 2/24 | 97 | 10 | 36/37 |

Table 3 shows the analysis and comparison of the various parameters used by the pathologist to characterize melanoma vs. the percent negative cells (negative for DUSP1). Clearly, the percent cells negative for DUSP1 has the highest Chi Square value, and the strongest correlation with overall survival is seen with the percent negative cells in melanoma.

TABLE 3
Comparison of Predictor variables for Death Using ROC and KM Curves

| | ROC | |
|---|---|---|
| Predictor | AUC | KM Chi Square |
| Clark level | .863 | 29.3 |
| Stage | .681 | 15.7 |
| Cox model (Clark, Cells o, Age) | .830 | 18.2 |
| Breslow | .883 | 13.4 (Cutoff 1.95) |
| Cells 3 | .674 | 7.4 (Cutoff 3.5) |
| Cells 2 | .852 | 26.4 (Cutoff 25) |
| Cells 1 | .626 | 3.5 (Cutoff 7.5) |
| Cells 0 | .869 | 38.6 (Cutoff 37.5) |
| Cells total (3 * Cells 3 + 2 * Cells 2 + Cells | .871 | 31.0 (Cutoff 103.5) |
| 1) | | |

As seen from Table 3, several of the predictors have good AUC in the ROC analysis. Six predictors have areas of at least 0.830. These curves were generated using the outcome of death but ignoring the time element. KM curves were analyzed based on survival data. When the natural strata for Clark level and Stage and the cutoff that has the highest sum of sensitivity and specificity for the continuous predictors were used, the highest Chi Square values and, therefore, the smallest p values were Clark level, Cells 2, Cells 0 and Cells Total. Cells 0 was significantly better than the next best predictor, Cells Total. Even through the Cox model using Clark level, Age and Cells 0, in which each of these predictors were significant, seemed like it should yield the best separation in a KM curve, the survival function from this model, using the best cutoff score, did not translate into a high Chi Square value in the KM analysis. It appeared that the best predictor of death (or survival) is the Cells 0 percentage, with the best cutoff 37.5 (sensitivity 0.095, specificity 0.875).

Table 4 shows the data for confirmed melanoma samples (below).

| | Patient | | | Procedure to |
|---|---|---|---|---|
| Diagnosis | Age | MKP-V15 | MKP-C19 | DOD(years) |
| metastatic melanoma | 35 | No data | No data | |
| metastatic melanoma | 52 | No data | No data | |
| recurrent melanoma of | 87 | (++)(Spindle Cell type) | (++) | |
| left foot | | | | |
| metastatic melanoma | 26 | (+) | (−) | |
| metastatic melanoma | 16 | (++) | (++) | |
| metastatic melanoma | 37 | No data | | |
| metastatic melanoma | 64 | | | |
| metastatic melanoma | 57 | No data | (−) | |
| metastatic melanoma | 25 | (++++) | (+++) | |
| metastatic melanoma | 59 | No data | No data | |
| metastatic melanoma | 48 | weak | (−) | |
| metastatic melanoma | 70 | (+++) | (+) | |
| metastatic melanoma | 41 | No data | No data | |
| metastatic melanoma | 87 | (+++) | (+++) | |
| metastatic melanoma | 49 | No data | No data | |
| metastatic melanoma | 70 | No data | No data | |
| to papillary dermis | | | | |
| metastatic melanoma | 59 | (+)/50%(−) | (+)/50%(−) | |
| metastatic melanoma | 58 | 50%(++), 50%(+) | No data | |
| metastatic melanoma | 75 | (+) | 50%(+), 50%(−) | |
| metastatic melanoma | 57 | (+) | 70%(−), 30%(+) | |
| metastatic melanoma | 43 | (+++) | (+++) | |
| metastatic melanoma | 32 | (++++) spindel cell tepy | (+++) | |
| metastatic melanoma | 66 | (+++) | 50%(++), 50%(+) | |
| metastatic melanoma | 78 | no data | no data | |
| metastatic melanoma | 69 | no dada | no data | |
| metastatic melanoma, | 18 | No data | No data | |
| however, on array no | | | | |
| tumor cells in section, | | | | |
| per Dr. Paul Duray | | | | |
| metastatic melanoma | 50 | Tu50%(+) | 70%(++)/30(+) | |
| metastatic melanoma | 28 | (+++) | (+) | |
| metastatic melanoma | 50 | (+) | (++) | |
| metastatic melanoma -- | 30 | (++++) | (+++) | |
| poorly differentiated | | | | |
| histology | | | | |
| metastatic melanoma | 58 | (++) | (++) | |
| metastatic melanoma -- | 33 | (++) | (++) | |
| epitheliod type | | | | |
| metastatic melanoma | 58 | (+) | (+) | |
| metastatic melanoma | 29 | No data | No data | |
| metastatic melanoma | 30 | (++) | (++) | |
| metastatic melanoma | 40 | (+++) | (+) | |
| metastatic melanoma | 21 | No data | No data | |
| metastatic melanoma | 37 | No data | No data | |
| metastatic melanoma | 30 | (+++) | (++) | |
| metastatic melanoma | 54 | No data | No data | |
| metastatic melanoma | 55 | (++) | No data | |
| metastatic melanoma | 60 | (++++) | (+++) | |
| metastatic melanoma | 61 | No data | No data | |
| primary melanoma | 22 | (++) junctional primary | (+) | |
| in situ | | melanoma | | |
| primary melanoma (0.6 | 65 | Weak(almost | Weak(almost | |
| mm Breslow thickness) | | nomal nevus) | nomal nevus) | |
| compound nevi | 51 | No data | No data | |
| basal cell carcinoma | 28 | No data | No data | |
| metastatic melanoma | 49 | (+)/weak | (−) | |
| poorly differentiated | 29 | No data | No data | |
| malignant neoplasm | | | | |
| consistent with | | | | |
| metastatic melanoma | | | | |
| metastatic melanoma | 49 | No data | No data | |
| metastatic melanoma | 41 | (+++) | (++) | |
| metastatic melanoma | 29 | (+) | (−) | |
| metastatic melanoma | 26 | (++) | (++) | |
| metastatic melanoma | 27 | (++++) Polymorphocellular | 50%(++), 50%(+) | |
| | | tumor | | |
| metastatic melanoma | 52 | 40%(++), 60%(+) spindle | 10%(+) 90%(−) | |
| | | cell type | | |
| metastatic melanoma | 20 | (+) | (+) | |
| metastatic melanoma | 33 | No data | No data | |
| (spindle cell type) | | | | |
| metastatic melanoma | 40 | (++++) | (++++) | |
| metastatic melanoma | 48 | No data | No data | |
| metastatic melanoma -- | 61 | (++++) | (++++) | |
| spindle fusiform | | | | |
| cell type | | | | |
| ocular melanoma with | 59 | (+++) | (++) | |
| liver metastasis | | | | |
| metastatic melanoma | 25 | (+++) | (+++) | |
| metastatic melanoma | 81 | (++++) | (++++) | |
| metastatic melanoma | 68 | 10% is tumor (+++) | No data | |
| metastatic melanoma | 25 | (+++) | (++) | |
| metastatic melanoma | 28 | (++) | (++) | |
| dysplastic nevi | 31 | (+++) | | |
| metastatic melanoma | 44 | No data | No data | |
| metastatic melanoma | 58 | No data | No data | |
| metastatic melanoma | 55 | No data | No data | |
| metastatic melanoma | 50 | (++++) | (++++) | |
| compound congenital | 21 | | | |
| nevi | | | | |
| metastatic melanoma | 29 | (+) | No data | |
| metastatic melanoma | 27 | (++++) | (+++) | |
| metastatic melanoma | 25 | No data | No data | |
| Primary melanoma | 22 | No data | No data | |
| metastatic melanoma | 21 | (+++) | No data | |
| metastatic melanoma | 30 | (+++) | (+++) | |
| metastatic melanoma | 23 | No data | No data | |
| metastatic melanoma | 47 | 50% is tumor (+) | 30% is tumor(+) | |
| with anaplastic tumor | | | | |
| giant cells and | | | | |
| sarcomatoid growth | | | | |
| pattern | | | | |
| metastatic melanoma | 58 | (+) | (+) | |
| metastatic melanoma | 49 | (++++) | (+++) | |
| metastatic melanoma | 38 | (++++) | (++) | |
| primary melanoma | 22 | No data | No data | |
| in situ | | | | |
| metastatic melanoma | 38 | (+++) | | |
| metastatic melanoma | 47 | (+++) | (++) | |
| metastatic melanoma | 37 | (+++) | (++) | |
| metastatic melanoma | 50 | (++++) | (++++) | |
| metastatic melanoma | 51 | (++++) | (++++) | |
| metastatic melanoma | 37 | (+++) | (++) | |
| metastatic melanoma | 31 | (++++) | (+++) | |
| primary melanoma | 29 | No data | No data | |
| in situ | | | | |
| metastatic melanoma | 25 | No data | No data | |
| Primary melanoma, | 14 | No data | No data | |
| radial phase | | | | |
| melnaoma Breslow | | | | |
| thickness 0.5 mm | | | | |
| metastatic melanoma | 16 | (+++) | (++) | |
| recurrent melanoma | 15 | (+++) | (+++) | |
| metastatic melanoma | 16 | (+++) | (++) | |
| metastatic melanoma, | 54 | 20% is tumor (++++) | No data | |
| epitheloid type | | | | |
| metastatic melanoma | 45 | (−) | (+)(−) | |
| spitz nevus | 14 | No data | No data | |
| metastatic melanoma | 50 | No data | No data | |
| metastatic melanoma | 51 | 40% is tumor(++) | 40% is tumor(++) | |
| metastatic melanoma | 33 | (++++) | (++) | |
| papillary dermal | 18 | No data | No data | |
| nevus | | | | |
| metastatic melanoma | 57 | (++++) | No data | |
| intradermal nevus, | 46 | No data | No data | |
| may represent a | | | | |
| congenital nevus | | | | |
| metastatic melanoma | 51 | (+++) | (+) | |
| intradermal nevus | 67 | | | |
| compound nevus | 37 | | | |
| recurrent melanoma | 68 | | | |
| metastatic melanoma | 64 | | | |
| metastatic melanoma | 60 | | | |
| metastatic melanoma | 34 | | | |
| metastatic melanoma | 42 | | | |
| metastatic melanoma | 51 | | | |
| metastatic melanoma | 41 | | | |
| metastatic melanoma | 38 | | | |
| metastatic melanoma | 51 | | | |
| metastatic melanoma | 49 | | | |
| metastatic melanoma | 33 | | | |
| recurrent melanoma | 82 | | | |
| metastatic melanoma | 17 | | | |
| recurrent melanoma | 29 | | | |
| recurrent melanoma | 60 | | | |
| metastatic melanoma | 17 | | | |
| metastatic melanoma | 56 | | | |
| metastatic melanoma | 56 | | | |
| metastatic melanoma | 18 | | | |
| metastatic melanoma | 42 | (++++) | (++) | |
| intradermal nevus | 50 | | | |
| intradermal nevus | 30 | (+) | No data | |
| intradermal nevus | 36 | | | |
| intradermal nevus | 69 | | | |
| intradermal nevus | 58 | | | |
| ntradermal nevus | 75 | | | |
| intradermal nevus | 39 | | | |
| intradermal nevus | 75 | | | |
| intradermal nevus | 49 | | | |
| intradermal nevus | 40 | | | |
| junctional nevus | 68 | | | |
| arising in association | | | | |
| with preexisting | | | | |
| benign lentigo | | | | |
| intradermal nevus | 13 | | | |
| intradermal nevus | 23 | | | |
| polypoid compound | 53 | | | |
| nevus | | | | |
| intradermal nevus | 41 | | | |
| junctional nevus | 25 | | | |
| compound nevus | 22 | | | |
| intradermal nevus | 38 | | | |
| intradermal nevus | 48 | | | |
| intradermal lesion | 38 | | | |
| compound nevus | 15 | | | |
| intradermal lesion | 42 | | | |
| compound nevus | 33 | | | |
| intradermal nevus | 58 | | | |
| intrademal nevus | 52 | | | |
| compound nevus | 39 | | | |
| papillary intradermal nevus | 49 | | | |
| intradermal neural nevus | 74 | | | |
| intraderma nevus | 30 | | | |
| intradermal nevus | 37 | | | |
| lentiginous compound | 29 | | | |
| nevus | | | | |
| intradermal nevus | 52 | | | |
| papillary intradermal nevus | unknown | | | |
| polypoid intradermal nevus | 44 | | | |
| intradermal nevus | 43 | (+++) | | |
| compound nevus | 47 | | | |
| compound nevus | 30 | | | |
| intradermal nevus | 38 | | | |
| blue nevus | 36 | | | |
| intradermal nevus | 71 | | | |
| junctional nevus | 55 | | | |
| compound nevus | 38 | | | |
| benign polypoid nevus | 15 | | | |
| lentiginous compound | 68 | | | |
| nevus | | | | |
| compound nevus | 36 | | | |
| congenital compound | 64 | | | |
| nevus | | | | |

| | Patient | Procedure | Date of | Array | Hospital |
|---|---|---|---|---|---|
| Diagnosis | Age | Date | Death | location | ID |
| metastatic melanoma | 35 | Dec. 15, 1995 | Jun. 14, 1997 | A12c | 2854922 |
| metastatic melanoma | 52 | Jul. 14, 1995 | Oct. 11, 1997 | A1h | 2834182 |
| recurrent melanoma of | 87 | Jun. 19, 1997 | Oct. 26, 1997 | A13e | 2682035 |
| left foot | | | | | |
| metastatic melanoma | 26 | Oct. 21, 1997 | Jan. 9, 2000 | A4e | 3074274 |
| metastatic melanoma | 16 | Mar. 31, 1997 | Alive | A4f | 3017898 |
| metastatic melanoma | 37 | Jul. 16, 1997 | Alive | A4g | 2966001 |
| metastatic melanoma | 64 | Sep. 16, 1999 | | Not on | 3239305 |
| | | | | array? | |
| metastatic melanoma | 57 | Apr. 20, 1998 | Jan. 8, 1999 | A4i | 2947134 |
| metastatic melanoma | 25 | Dec. 9, 1994 | Jul. 28, 1995 | A5a | 2769189 |
| metastatic melanoma | 59 | Jul. 21, 1995 | Jan. 31, 1998 | A1i | 2791407 |
| metastatic melanoma | 48 | Jun. 3, 1997 | Alive | A3e | 3073178 |
| metastatic melanoma | 70 | Aug. 13, 1997 | May 3, 1998 | A2e | 3100339 |
| metastatic melanoma | 41 | Dec. 17, 1997 | Sep. 17, 2000 | A12d | 3138458 |
| metastatic melanoma | 87 | May 1, 1995 | Unknown | A12e | 2636645 |
| metastatic melanoma | 49 | Jun. 9, 1997 | Dec. 15, 2000 | A12f | 2885748 |
| metastatic melanoma | 70 | Dec. 17, 1997 | Alive | A12g | 3074079 |
| to papillary dermis | | | | | |
| metastatic melanoma | 59 | Jan. 28, 1998 | Oct. 28, 1998 | A12h | 3022791 |
| metastatic melanoma | 58 | May 5, 1995 | Nov. 23, 1996 | A12i | 2742573 |
| metastatic melanoma | 75 | Sep. 14, 1995 | Feb. 20, 1996 | A13a | 2735787 |
| metastatic melanoma | 57 | Nov. 12, 1997 | Dec. 11, 2000 | A13b | 3122992 |
| metastatic melanoma | 43 | Jun. 13, 1997 | Alive | A13c | 2685632 |
| metastatic melanoma | 32 | Jun. 20, 1995 | Alive | A13d | 2226169 |
| metastatic melanoma | 66 | May 13, 1997 | Jun. 10, 1998 | A13f | 2773296 |
| metastatic melanoma | 78 | May 6, 1997 | Mar. 14, 1999 | A13g | 3068961 |
| metastatic melanoma | 69 | Nov. 13, 1995 | Jul. 6, 1998 | A1f | 2721259 |
| metastatic melanoma, | 18 | Apr. 30, 1997 | Unknown | A2a | 3066319 |
| however, on array no | | | | | |
| tumor cells in section, | | | | | |
| per Dr. Paul Duray | | | | | |
| metastatic melanoma | 50 | Mar. 5, 1998 | Nov. 18, 1998 | A2b | 3147162 |
| metastatic melanoma | 28 | Aug. 29, 1994 | Unknown | A2d | 2797549 |
| metastatic melanoma | 50 | May 16, 1997 | Jan. 17, 1998 | A2g | 2999924 |
| metastatic melanoma -- | 30 | Nov. 7, 1995 | Oct. 30, 1996 | A2h | 2885761 |
| poorly differentiated | | | | | |
| histology | | | | | |
| metastatic melanoma | 58 | Aug. 24, 1995 | Jan. 21, 1997 | A2i | 2787350 |
| metastatic melanoma -- | 33 | Jan. 28, 1998 | Feb. 3, 2000 | A3a | 3087347 |
| epitheliod type | | | | | |
| metastatic melanoma | 58 | May 16, 1995 | Apr. 6, 1997 | A3b | 2858411 |
| metastatic melanoma | 29 | Nov. 9, 1994 | May 25, 1995 | A3g | 2812435 |
| metastatic melanoma | 30 | Mar. 10, 1994 | Alive | A3h | 2750570 |
| metastatic melanoma | 40 | Feb. 21, 1995 | Unknown | A3i | 2842804 |
| metastatic melanoma | 21 | Feb. 10, 1993 | Feb. 5, 1994 | A4a | 2522469 |
| metastatic melanoma | 37 | Jun. 17, 1997 | Nov. 2, 1997 | A4b | 3051912 |
| metastatic melanoma | 30 | Sep. 3, 1993 | Dec. 10, 1996 | A4c | 2461766 |
| metastatic melanoma | 54 | Oct. 17, 1997 | Unknown | A4d | 3113176 |
| metastatic melanoma | 55 | Mar. 18, 1998 | Jun. 27, 1999 | B2f | 3112639 |
| metastatic melanoma | 60 | Nov. 20, 1995 | Dec. 9, 1997 | B3a | 2779237 |
| metastatic melanoma | 61 | Feb. 8, 1997 | Alive | A11b | 1467426 |
| primary melanoma | 22 | Sep. 29, 1995 | Unknown | A7c | 1748063 |
| in situ | | | | | |
| primary melanoma (0.6 | 65 | Nov. 14, 1995 | Alive | A8a | 2057578 |
| mm Breslow thickness) | | | | | |
| compound nevi | 51 | Aug. 4, 1997 | Unknown | B6g | 1313113 |
| basal cell carcinoma | 28 | Mar. 3, 1997 | Alive | B6h | 1835269 |
| metastatic melanoma | 49 | Aug. 1, 1997 | Dec. 15, 2000 | B7e | 2885748 |
| poorly differentiated | 29 | Aug. 17, 1993 | Dec. 4, 1993 | B7f | 2695613 |
| malignant neoplasm | | | | | |
| consistent with | | | | | |
| metastatic melanoma | | | | | |
| metastatic melanoma | 49 | Sep. 30, 1997 | Dec. 15, 2000 | B7h | 2885748 |
| metastatic melanoma | 41 | Oct. 2, 1998 | Oct. 8, 1999 | B7i | 3138628 |
| metastatic melanoma | 29 | May 11, 1992 | Oct. 18, 1992 | B8a | 2519343 |
| metastatic melanoma | 26 | Jul. 30, 1997 | Jan. 9, 2000 | A12a | 3074274 |
| metastatic melanoma | 27 | Oct. 5, 1992 | Mar. 20, 1993 | B1a | 2597196 |
| metastatic melanoma | 52 | Mar. 1l, 1997 | Jan. 16, 1998 | B1b | 3025160 |
| metastatic melanoma | 20 | Sep. 30, 1993 | Dec. 1, 1993 | A10f | 2703646 |
| metastatic melanoma | 33 | May 28, 1997 | Unknown | B1d | 3070384 |
| (spindle cell type) | | | | | |
| metastatic melanoma | 40 | Mar. 30, 1998 | Alive | B1f | 3036741 |
| metastatic melanoma | 48 | Jan. 28, 1998 | Alive | B1i | 3064104 |
| metastatic melanoma -- | 61 | Aug. 1, 1995 | Alive | B2b | 2887186 |
| spindle fusiform | | | | | |
| cell type | | | | | |
| ocular melanoma with | 59 | Dec. 21, 1995 | Unknown | B2c | 2919771 |
| liver metastasis | | | | | |
| metastatic melanoma | 25 | Jan. 20, 1994 | Jun. 22, 1994 | B2d | 2738727 |
| metastatic melanoma | 81 | May 16, 1995 | Unknown | B1e | 2861045 |
| metastatic melanoma | 68 | Jul. 2, 1997 | Alive | B3c | 3081874 |
| metastatic melanoma | 25 | Jul. 9, 1999 | Alive | B9e | 3018209 |
| metastatic melanoma | 28 | Sep. 30, 1996 | Nov. 15, 1998 | B3f | 2979196 |
| dysplastic nevi | 31 | Nov. 30, 1997 | Alive | B10c | 2612938 |
| metastatic melanoma | 44 | Mar. 16, 1998 | alive | B8e | 2685632 |
| metastatic melanoma | 58 | Apr. 8, 1998 | Jan. 8, 1999 | B8f | 2947134 |
| metastatic melanoma | 55 | Jul. 29, 1997 | Alive | B10i | 3089757 |
| metastatic melanoma | 50 | Sep. 23, 1999 | Unknown | B2e | 2925230 |
| compound congenital | 21 | Oct. 19, 1999 | Unknown | B10e | X000002823 |
| nevi | | | | | |
| metastatic melanoma | 29 | Oct. 5, 1992 | Unknown | C8b | 2226169 |
| metastatic melanoma | 27 | Feb. 12, 1993 | Unknown | C5d | 2584372 |
| metastatic melanoma | 25 | Aug. 26, 1993 | Unknown | C11d | 2695613 |
| Primary melanoma | 22 | Feb. 28, 1994 | Alive | C11c | 1835269 |
| metastatic melanoma | 21 | Mar. 3, 1994 | Unknown | B12a | 2728412 |
| metastatic melanoma | 30 | Mar. 25, 1994 | Unknown | C1h | Unknown |
| metastatic melanoma | 23 | Jun. 29, 1994 | Unknown | B11c | 2776765 |
| metastatic melanoma | 47 | Jan. 6, 1995 | Unknown | C2g | 2827657 |
| with anaplastic tumor | | | | | |
| giant cells and | | | | | |
| sarcomatoid growth | | | | | |
| pattern | | | | | |
| metastatic melanoma | 58 | Jan. 12, 1995 | Unknown | C7a | 2800056 |
| metastatic melanoma | 49 | Jan. 13, 1995 | Unknown | C1c | 2836543 |
| metastatic melanoma | 38 | Feb. 14, 1995 | Unknown | B13a | 2773235 |
| primary melanoma | 22 | Feb. 17, 1995 | Unknown | A7e | 1748063 |
| in situ | | | | | |
| metastatic melanoma | 38 | Mar. 21, 1995 | Unknown | A10h | 2760423 |
| metastatic melanoma | 47 | Mar. 31, 1995 | Unknown | A9d | 2850643 |
| metastatic melanoma | 37 | May 17, 1995 | Unknown | C2h | 2848958 |
| metastatic melanoma | 50 | Jun. 16, 1995 | Unknown | A10g | 2532694 |
| metastatic melanoma | 51 | Jul. 10, 1995 | Unknown | A9i | 2869469 |
| metastatic melanoma | 37 | Jul. 26, 1995 | Unknown | C5b | 2598139 |
| metastatic melanoma | 31 | Aug. 22, 1995 | Unknown | C7f | 2815837 |
| primary melanoma | 29 | Sep. 11, 1995 | Unknown | A7b | 2335451 |
| in situ | | | | | |
| metastatic melanoma | 25 | Feb. 13, 1996 | Unknown | C6d | 2917968 |
| Primary melanoma, | 14 | Aug. 7, 1996 | Unknown | A8e | 1884438 |
| radial phase | | | | | |
| melnaoma Breslow | | | | | |
| thickness 0.5 mm | | | | | |
| metastatic melanoma | 16 | Oct. 23, 1996 | Unknown | A10c | 2971616 |
| recurrent melanoma | 15 | Nov. 14, 1996 | Alive | B9h | 3017898 |
| metastatic melanoma | 16 | Dec. 5, 1996 | Unknown | C2a | 2971616 |
| metastatic melanoma, | 54 | Mar. 7, 1997 | Unknown | C12e | 2986851 |
| epitheloid type | | | | | |
| metastatic melanoma | 45 | Apr. 16, 1997 | Dec. 1, 1998 | C2i | 3040707 |
| spitz nevus | 14 | May 8, 1997 | Unknown | C3h | 3071947 |
| metastatic melanoma | 50 | May 22, 1997 | Unknown | C5g | 3072940 |
| metastatic melanoma | 51 | Jun. 12, 1997 | Unknown | B8h | 3019597 |
| metastatic melanoma | 33 | Jun. 13, 1997 | Unknown | C7i | 3078590 |
| papillary dermal | 18 | Jul. 11, 1997 | Unknown | B10g | 2201926 |
| nevus | | | | | |
| metastatic melanoma | 57 | Sep. 24, 1997 | Unknown | A2c | 2947134 |
| intradermal nevus, | 46 | Oct. 23, 1997 | Unknown | B11h | 3115951 |
| may represent a | | | | | |
| congenital nevus | | | | | |
| metastatic melanoma | 51 | Oct. 23, 1997 | Unknown | B9g | 2714280 |
| intradermal nevus | 67 | Nov. 14, 1997 | Unknown | C3g | 3123546 |
| compound nevus | 37 | May 14, 1997 | Unknown | A8h | 3058621 |
| recurrent melanoma | 68 | Jan. 23, 1998 | Unknown | B9b | 2787428 |
| metastatic melanoma | 64 | Jan. 30, 1998 | Unknown | C6i | 3107516 |
| metastatic melanoma | 60 | Feb. 18, 1998 | Unknown | B9i | 2988987 |
| metastatic melanoma | 34 | Feb. 20, 1998 | Unknown | B9c | 3061449 |
| metastatic melanoma | 42 | Feb. 26, 1998 | Unknown | B11a | 2954631 |
| metastatic melanoma | 51 | Mar. 17, 1998 | Unknown | C4b | 3080481 |
| metastatic melanoma | 41 | Mar. 26, 1998 | Unknown | C8g | 2598139 |
| metastatic melanoma | 38 | Mar. 27, 1998 | Unknown | C8i | 3077172 |
| metastatic melanoma | 51 | Apr. 1, 1998 | Unknown | A9f | 3080481 |
| metastatic melanoma | 49 | Apr. 15, 1998 | Unknown | C6e | 3170172 |
| metastatic melanoma | 33 | Apr. 23, 1998 | Unknown | A11a | 3077421 |
| recurrent melanoma | 82 | Apr. 29, 1998 | Unknown | C4c | 3078991 |
| metastatic melanoma | 17 | May 22, 1998 | Alive | B9d | 3017898 |
| recurrent melanoma | 29 | Jun. 5, 1998 | Unknown | C5e | 3043769 |
| recurrent melanoma | 60 | Jul. 24, 1998 | Unknown | A12b | 3193585 |
| metastatic melanoma | 17 | Aug. 3, 1998 | Alive | C5i | 3017898 |
| metastatic melanoma | 56 | Sep. 29, 1998 | Sep. 14, 1999 | C7g | 3221969 |
| metastatic melanoma | 56 | Dec. 11, 1998 | Sep. 14, 1999 | A9e | 3221969 |
| metastatic melanoma | 18 | Jul. 7, 1999 | Alive | B8c | 3017898 |
| metastatic melanoma | 42 | Aug. 25, 1999 | Unknown | C1g | 3314972 |
| | | Aug. 26, 1999 | Unknown | Not on | 3320960 |
| | | | | array? | |
| intradermal nevus | 50 | | | | |
| intradermal nevus | 30 | | | A5c | |
| intradermal nevus | 36 | | | A5b | |
| intradermal nevus | 69 | | | A5d | |
| intradermal nevus | 58 | | | A5e | |
| ntradermal nevus | 75 | | | A5f | |
| intradermal nevus | 39 | | | A6d | |
| intradermal nevus | 75 | | | A5h | |
| intradermal nevus | 49 | | | A5g | |
| intradermal nevus | 40 | | | A5i | |
| junctional nevus | 68 | | | A1a | |
| arising in association | | | | | |
| with preexisting | | | | | |
| benign lentigo | | | | | |
| intradermal nevus | 13 | | | A6f | |
| intradermal nevus | 23 | | | A6a | |
| polypoid compound | 53 | | | A6b | |
| nevus | | | | | |
| intradermal nevus | 41 | | | B3h | |
| junctional nevus | 25 | | | A11d | |
| compound nevus | 22 | | | A6g | |
| intradermal nevus | 38 | | | A6i | |
| intradermal nevus | 48 | | | A1b | |
| intradermal lesion | 38 | | | A1c | |
| compound nevus | 15 | | | A11e | |
| intradermal lesion | 42 | | | B3i | |
| compound nevus | 33 | | | B5a | |
| intradermal nevus | 58 | | | B4a | |
| intrademal nevus | 52 | | | B4b | |
| compound nevus | 39 | | | B5b | |
| papillary intradermal nevus | 49 | | | B4c | |
| intradermal neural nevus | 74 | | | B4d | |
| intraderma nevus | 30 | | | B5d | |
| intradermal nevus | 37 | | | B4e | |
| lentiginous compound | 29 | | | A11f | |
| nevus | | | | | |
| intradermal nevus | 52 | | | B4f | |
| papillary intradermal nevus | unknown | | | B4g | |
| polypoid intradermal nevus | 44 | | | B4h | |
| | | | | Not on | |
| | | | | array? | |
| intradermal nevus | 43 | | | C9a | |
| compound nevus | 47 | | | B5g | |
| compound nevus | 30 | | | B5h | |
| intradermal nevus | 38 | | | C9d | |
| blue nevus | 36 | | | C10e | |
| intradermal nevus | 71 | | | C9e | |
| junctional nevus | 55 | | | Not on | |
| | | | | arrray? | |
| compound nevus | 38 | | | A1d | |
| benign polypoid nevus | 15 | | | C9f | |
| lentiginous compound | 68 | | | C9g | |
| nevus | | | | | |
| compound nevus | 36 | | | A11g | |
| congenital compound | 64 | | | A11h | |
| nevus | | | | | |

All publications, patents, and patent applications mentioned in this specification are herein incorporated by reference to the same extent as if each individual publication, patent, or patent application was specifically and individually indicated to be incorporated by reference.

The present disclosure has been described above with reference to exemplary embodiments. However, those skilled in the art, having read this disclosure, will recognize that changes and modifications may be made to the exemplary embodiments without departing from the scope of the present disclosure. For example, other types of immunostaining may be employed to detect expression of DUSP1. These and other changes or modifications are intended to be included within the scope of the present disclosure, as expressed in the following claims.

What is claimed is:

1. A method of screening for a skin cancer in a subject which comprises detecting an expression level of dual specificity phosphatase 1 protein (DUSP1) in the subject and comparing the expression level of DUSP1 to a level of DUSP1 expression predetermined to correlate with the presence or absence of the skin cancer.

2. The method of claim 1, wherein the skin cancer is basal cell carcinoma, squamous cell carcinoma, melanoma, dermatofibrosarcoma protuberans, Merkel cell carcinoma, neuroectodermal tumor, lympathic epithelial tumor or sarcoma Kaposi.

3. The method of claim 2, wherein melanoma is selected from the group consisting of malignant melanoma, cutaneous melanoma and intraocular melanoma.

4. The method of claim 2, wherein melanoma is an early stage melanoma.

5. The method of claim 1, wherein the expression level of DUSP1 is detected by protein detection technique.

6. The method of claim 5, wherein the protein detection technique is selected from the group consisting of western blott, protein array, immunohistochemical assay, ELISA, fluorescent-activated cell sorting, flow: cytometry, and mass spectrometry.

7. The method of claim 6, wherein the protein detection technique is immunohistochemical staining.

8. A method for prognosis of survivability of a subject having a skin cancer comprising detecting an expression level of DUSP1 in the subject and comparing the expression level of DUSP1 to an expression level index of DUSP1 predetermined to correlate with survivability.

9. The method of claim 8, wherein the predetermined DUSP1 expression level index is obtained by assaying skin cancer samples from subjects with the same type of skin cancer for the expression of DUSP1 and correlating the DUSP1 expression levels with the observed survivability of the subjects.

10. The method, of claim 9, wherein the patients' survivability corresponds to a percentage of DUSP1 expressing cells on the predetermined DUSP1 expression level index.

11. The method of claim 9, wherein the DUSP1 expression level index is determined by assaying the percentage of cells in a skin cancer sample that are positive for DUSP1 expression, wherein the DUSP1 expression level index values that correlate with increased survivability range from about 5% to about 100%.

12. The method of claim 11, wherein the range is from about 25% to about 100%.

13. The method of claim 12, wherein the range is from about 35% to about 100%.

14. The method of claim 8, wherein the DUSP1 expression level index is determined by assaying the percentage of cells in a skin cancer sample that are positive for DUSP1 expression, and categorizing the positive cells into semi-quantitative levels of expression based on the intensity of the staining, wherein the range of staining is from 0 to 3.

15. The method of claim 9, wherein the DUSP1 expression level index is determined by assaying the percentage of cells in a skin cancer sample that are negative for DUSP1 expression, wherein the DUSP1 expression level index values that correlate with reduced survivability range from about 5% to about 100%.

16. The method of claim 15, wherein the range is from about 25% to about 100%.

17. The method of claim 16, wherein the range is from about 35% to about 100%.

18. The method of claim 8, wherein the skin cancer is basal cell carcinoma, squamous cell carcinoma, melanoma, dermatofibrosarcoma protuberans, Merkel cell carcinoma or sarcoma Kaposi.

19. The method of claim 18, wherein the skin cancer is melanoma.

20. The method of claim 19, wherein melanoma is an early stage melanoma.

21. The method of claim 8, wherein the expression level of DUSP1 is detected by protein detection technique.

22. The method of claim 21, wherein the protein detection technique is immunohistochemical staining.

23. A method of treating a skin cancer comprising administering to a patient in need of such treatment an effective amount of DUSP1 or an active fragment thereof, a nucleic acid that encodes a protein comprising DUSP1 or an active fragment, an analog or a prodrug thereof.

24. The method of claim 23, further comprising administering to a patient an agent that increases the expression of DUSP1, wherein the agent is angiotensin, aldosterone, AG1478 or U0126.

25. A pharmaceutical composition for treating a skin cancer comprising a DUSP1 or an active fragment thereof, a nucleic acid that encodes a protein comprising DUSP1 or an active fragment, an analog or a prodrug thereof, and a pharmaceutically acceptable carrier, excipient or diluent.

26. The pharmaceutical composition of claim 25, further comprising an agent that increases the expression of DUSP1, wherein the agent is angiotensin, aldosterone, AG1478 or U0126.

27. A kit comprising a reagent for detection of DUSP1 expression level, a compilation comprising DUSP1 expression level indices that have been predetermined to correlate with the presence or absence of skin cancer, assay components and components for collection of a sample.

28. A kit comprising a reagent for detection of DUSP1 expression level, a compilation comprising DUSP1 expression level indices that have been predetermined to correlate with cancer survivability, assay components and components for collection of a sample.
